SEQUENCE LISTING (33 sequences)

SEQ ID NO: 1 - protein, 10 aa, Mus musculus
GDAFTNYLIE

SEQ ID NO: 2 - protein, 17 aa, Mus musculus
LIYPDSGYINYNENFKG

SEQ ID NO: 3 - protein, 13 aa, Mus musculus
RFAYYGSGYYFDY

SEQ ID NO: 4 - protein, 16 aa, Mus musculus
RSSQSLLKTNGNTYLH

SEQ ID NO: 5 - protein, 7 aa, Mus musculus
KVSNRFS

SEQ ID NO: 6 - protein, 9 aa, Mus musculus
SQSTHFPFT

SEQ ID NO: 7 - protein, 5 aa, Mus musculus
NYLIE

SEQ ID NO: 8 - protein, 10 aa, Mus musculus
GYGFINYLIE

SEQ ID NO: 9 - protein, 17 aa, Mus musculus
LINPGSDYTNYNENFKG

SEQ ID NO: 10 - protein, 13 aa, Mus musculus
RFGYYGSGNYFDY

SEQ ID NO: 11 - protein, 16 aa, Mus musculus
TSGQSLVHINGNTYLH

SEQ ID NO: 12 - protein, 7 aa, Mus musculus
KVSNLFS

SEQ ID NO: 13 - protein, 17 aa, Mus musculus
LIIPGTGYTNYNENFKG

SEQ ID NO: 14 - protein, 13 aa, Mus musculus
RFGYYGSSNYFDY

SEQ ID NO: 15 - protein, 16 aa, Mus musculus
RSSQSLVHSNGNTYLH

SEQ ID NO: 16 - protein, 13 aa, Mus musculus
RFGYYGSGYYFDY

SEQ ID NO: 17 - protein, 11 aa, Mus musculus
GFSITSGYYWT

SEQ ID NO: 18 - protein, 16 aa, Mus musculus
YIGYDGSNDSNPSLKN

SEQ ID NO: 19 - protein, 9 aa, Mus musculus
AMLRRGFDY

SEQ ID NO: 20 - protein, 10 aa, Mus musculus
SASSSLSYMH

SEQ ID NO: 21 - protein, 7 aa, Mus musculus
DTSKLAS

SEQ ID NO: 22 - protein, 7 aa, Mus musculus
HRRSSYT

SEQ ID NO: 23 - protein, 6 aa, Mus musculus
SGYYWT

SEQ ID NO: 24 - protein, 122 aa, Mus musculus
QVKLQQSGPELVRPGTSVKVSCTASGDAFTNYLIEWVKQRPGQGLEWIGLIYPDSGYINYNENFKGKATLTADRSSSTAYMQLSSLTSEDSAVYFCARRFAYYGSGYYFDYWGQGTTLTVSS

SEQ ID NO: 25 - protein, 112 aa, Mus musculus
DVVMTQTPLSLPVSLGDQASISCRSSQSLLKTNGNTYLHWYLQKPGQSPKLLIFKVSNRFSGVPDRFSGSGSGTDFTLKISRVEAEDLGVYFCSQSTHFPFTFGTGTKLEIK

SEQ ID NO: 26 - protein, 122 aa, Mus musculus
QVQLQQSGAELVRPGTSVKVSCKASGYGFINYLIEWIKQRPGQGLEWIGLINPGSDYTNYNENFKGKATLTADKSSSTAYMHLSSLTSEDSAVYFCARRFGYYGSGNYFDYWGQGTTLTVSS

SEQ ID NO: 27 - protein, 112 aa, Mus musculus
DVVMTQTPLSLPVSLGDQASISCTSGQSLVHINGNTYLHWYLQKPGQSPKLLIYKVSNLFSGVPDRFSGSGSGTDFTLKISRVEAEDLGVYFCSQSTHFPFTFGTGTKLEIK

SEQ ID NO: 28 - protein, 122 aa, Mus musculus
QVQLQQSGAELVRPGTSVKVSCKASGDAFTNYLIEWVKQRPGQGLEWIGLIIPGTGYTNYNENFKGKATLTADKSSSTAYMQLSSLTSEDSAVYFCARRFGYYGSSNYFDYWGQGTTLTVSS

SEQ ID NO: 29 - protein, 112 aa, Mus musculus
DVVMTQTPLSLPVSLGDQASISCRSSQSLVHSNGNTYLHWYLQKPGQSPKLLIYKVSNRFSGVPDRFSGSGPGTDFTLKISRVEAEDLGIYFCSQSTHFPFTFGTGTKLEIK

SEQ ID NO: 30 - protein, 122 aa, Mus musculus
QVQLQQSGAELVRPGTSVKLSCKASGDAFTNYLIEWVKQRPGQGLEWIGLIIPGTGYTNYNENFKGKATLTADKSSSTAYMQLSSLTSEDSAVYFCARRFGYYGSGYYFDYWGQGTTLTVSS

SEQ ID NO: 31 - protein, 112 aa, Mus musculus
DVVMTQTPLSLPVSLGDQASISCRSSQSLVHSNGNTYLHWYLQKPGQSPKLLIYKVSNRFSGVPDRFSGSGPGTDFTLKISRVEAEDLGVYFCSQSTHFPFTFGTGTKLEIK

SEQ ID NO: 32 - protein, 118 aa, Mus musculus
DIQLQESGPGLVKPSQSLSLTCSVTGFSITSGYYWTWIRQFPGNKLEWVAYIGYDGSNDSNPSLKNRISITRDTSKNQFFLKLNSVTTEDTATYYCARAMLRRGFDYWGQGTTLTVSS

SEQ ID NO: 33 - protein, 104 aa, Mus musculus
QIVLTQSPAIMSASPGEKVTMTCSASSSLSYMHWYQQKPGTSPKRWIYDTSKLASGVPARFSGSGSGTSYSLTISSMEAEDAATYYCHRRSSYTFGGGTKLEIK

(19) United States
(12) Patent Application Publication
(10) Pub. No.: US 2017/0114125 A1
(43) Pub. Date: Apr. 27, 2017

(54) METHODS OF TREATING DISEASE USING ANTIBODIES TO LYSOPHOSPHATIDIC ACID

(71) Applicants: Roger A. SABBADINI, Bend, OR (US); Rosalia MATTEO, Chula Vista, CA (US); Genevieve HANSEN, San Diego, CA (US); William Arthur GARLAND, San Clemente, CA (US); James Stephen SWANEY, Carlsbad, CA (US)

(72) Inventors: Roger A. SABBADINI, Bend, OR (US); Rosalia MATTEO, Chula Vista, CA (US); Genevieve HANSEN, San Diego, CA (US); William Arthur GARLAND, San Clemente, CA (US); James Stephen SWANEY, Carlsbad, CA (US)

(21) Appl. No.: 15/331,881

(22) Filed: Oct. 23, 2016

Related U.S. Application Data
(63) Continuation of application No. 12/836,524, filed on Jul. 14, 2010, now abandoned.
Application No. 12/836,524 is a continuation-in-part of application No. 12/406,874, filed on Mar. 18, 2009, now Pat. No. 9,163,091.
Application No. 12/406,874 is a continuation-in-part of application No. 12/129,109, filed on May 29, 2008, now Pat. No. 8,158,124.
Application No. 12/836,524 is a continuation-in-part of application No. 12/761,584, filed on Apr. 16, 2010, now Pat. No. 8,604,172.
(60) Provisional application No. 60/940,964, filed on May 30, 2007.

(51) Int. Cl. C07K 16/18; G01N 33/574; G01N 33/92

(52) U.S. Cl. CPC C07K 16/18; G01N 33/92; G01N 33/574; C07K 2317/565; C07K 2317/76; G01N 2405/04; A61K 2039/505

(57) ABSTRACT

Methods for preventing or treating pain are provided, comprising administering to a subject, including a human subject, an antibody or antibody fragment that binds LPA.

### RELATED APPLICATIONS

This application is a continuation of U.S. patent application Ser. No. 12/836,524 (attorney docket no. LPT-3200-CP2), filed 14 Jul. 2010, which is a continuation-in-part of U.S. patent application Ser. No. 12/406,874 (attorney docket no. LPT-3200-CP), filed 18 Mar. 2009, which in turn is a continuation-in-part of U.S. patent application Ser. No. 12/129,109 (attorney docket no. LPT-3200-UT), filed 28 May 2008, which in turn claims the benefit of and priority to U.S. provisional patent application Ser. No. 60/940,964 (attorney docket no. LPT-3200-PV), filed 30 May 2007. U.S. patent application Ser. No. 12/836,524 is also a continuation-in-part of U.S. patent application Ser. No. 12/761,584, filed 16 Apr. 2010 (attorney docket no. LPT-3210-UT). All of the above are commonly owned with the instant application and are herein incorporated by reference in their entirety and for all purposes, including continuity and benefit of priority.

### SEQUENCE LISTING

The instant application contains a sequence listing which has been submitted via EFS-Web and is hereby incorporated by reference in its entirety. The ASCII copy of the sequence listing, created on Oct. 23, 2016, is named LPT3200CP2CT.txt, and is 14,943 bytes in size.

### TECHNICAL FIELD

The present invention relates to agents that bind lysophosphatidic acid (LPA) and its variants, particularly to monoclonal antibodies, antibody fragments, and antibody derivatives specifically reactive to LPA under physiological conditions. Such agents can be used in the treatment and/or prevention of various diseases or disorders through the delivery of pharmaceutical compositions that contain such agents.

LPA is a bioactive lipid mediating multiple cellular responses including proliferation, differentiation, angiogenesis, motility, and protection from apoptosis in a variety of cell types.

LPA is involved in the establishment and progression of cancer by providing a pro-growth tumor microenvironment and promoting angiogenesis. In addition, LPA has been implicated in fibrosis, ocular diseases such as macular degeneration, and pain-related disorders. Therefore, an antibody-based approach to the neutralization of LPA offers the potential to increase the arsenal of current therapies for these indications.

The assignee has invented a family of high-affinity, specific monoclonal antibodies to LPA, one of which is known as Lpathomab or LT3000. The efficacy of Lpathomab in various animal models of cancer, fibrosis, and ocular disorders highlights the utility of this class of anti-LPA antibodies (and molecules derived therefrom), for example, in the treatment of malignancies, angiogenesis, and fibrosis-related disorders.

### BACKGROUND OF THE INVENTION

### 1. Introduction

The following description includes information that may be useful in understanding the present invention. It is not an admission that any of the information provided herein, or any publication specifically or implicitly referenced herein, is prior art, or even particularly relevant, to the presently claimed invention.

### 2. Background

A. Bioactive Signaling Lipids

Certain lipids and their derivatives are now recognized as important targets for medical research, not as just simple structural elements in cell membranes or as a source of energy for β-oxidation, glycolysis or other metabolic processes. In particular, certain lipids function as signaling mediators important in animal and human disease. Although most of the lipids of the plasma membrane play an exclusively structural role, a small proportion of them are involved in relaying extracellular stimuli into cells. These lipids are referred to as “bioactive lipids” or, alternatively, “bioactive signaling lipids.” “Lipid signaling” refers to any of a number of cellular signal transduction pathways that use cell membrane lipids as second messengers, as well as referring to direct interaction of a lipid signaling molecule with its own specific receptor. Lipid signaling pathways are activated by a variety of extracellular stimuli, ranging from growth factors to inflammatory cytokines, and regulate cell fate decisions such as apoptosis, differentiation and proliferation. Research into bioactive lipid signaling is an area of intense scientific investigation as more and more bioactive lipids are identified and their actions characterized.

Examples of bioactive lipids include the eicosanoids (including the cannabinoids, leukotrienes, prostaglandins, lipoxins, epoxyeicosatrienoic acids, and isoeicosanoids), non-eicosanoid cannabinoid mediators, phospholipids and their derivatives such as phosphatidic acid (PA) and phosphatidylglycerol (PG), platelet activating factor (PAF) and cardiolipins as well as lysophospholipids such as lysophosphatidyl choline (LPC) and various lysophosphatidic acids (LPA). Bioactive signaling lipids also include the sphingolipids such as sphingomyelin, ceramide, ceramide-1-phosphate, sphingosine, sphingosylphosphoryl choline, sphinganine, sphinganine-1-phosphate (dihydro-S1P) and sphingosine-1-phosphate. Sphingolipids and their derivatives represent a group of extracellular and intracellular signaling molecules with pleiotropic effects on important cellular processes. Other examples of bioactive signaling lipids include phosphatidylinositol (PI), phosphatidylethanolamine (PEA), diacylglyceride (DG), sulfatides, gangliosides, and cerebrosides.

1. Lysolipids

Lysophospholipids (LPLs), also known as lysolipids, are low molecular weight (typically less than about 500 dalton) lipids that contain a single hydrocarbon backbone and a polar head group containing a phosphate group. Some lysolipids are bioactive signaling lipids. Two particular examples of medically important bioactive lysolipids are LPA (glycerol backbone) and S1P (sphingoid backbone). The structures of selected LPAs, S1P, and dihydro S1P are presented below.

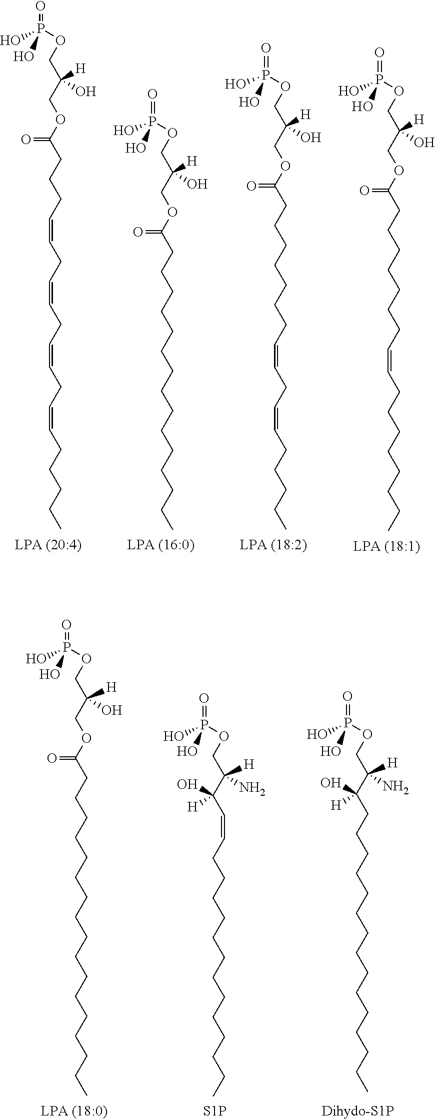

The structural backbone of LPA is derived from glycerol-based phospholipids such as phosphatidylcholine (PC) or phosphatidic acid (PA). In the case of lysosphingolipids such as SIP, the fatty acid of the ceramide backbone is missing. The structural backbone of SIP, dihydro SIP (DHS1P), and sphingosylphosphorylcholine (SPC) is based on sphingosine, which is derived from sphingomyelin.

LPA and SIP regulate various cellular signaling pathways by binding to the same class of multiple transmembrane domain G protein-coupled (GPCR) receptors. The SIP receptors are designated as S1P1, S1P2, S1P3, S1P4 and SIPS (formerly EDG-1, EDG-5/AGR16, EDG-3, EDG-6 and EDG-8) and the LPA receptors designated as LPA1, LPA2, LPA3 (formerly, EDG-2, EDG-4, and EDG-7). A fourth LPA receptor of this family has been identified for LPA (LPA4), and other putative receptors for these lysophospholipids have also been reported.

LPA and SIP have been shown to play a role in the immune response through modulation of immune-related cells such as T- and B-lymphocytes. These lipids promote T-cell migration to sites of immune response and regulate proliferation of T cells as well as secretion of various cytokines. In particular, SIP is thought to control egress of lymphocytes into the peripheral circulation. Thus agents which bind LPA and SIP are believed to be useful in methods for decreasing an undesired, excessive or aberrant immune response, and for treating diseases and conditions, including certain hematological cancers and autoimmune disorders that are associated with an undesired, excessive or aberrant involvement of lymphocytes and or an aberrant immune response.

a. Lysophosphatic Acid (LPA)

Lysophosphatidic acid (mono-acylglycerol-3-phosphate, <500 Dalton) consists of a single hydrocarbon backbone and a polar head group containing a phosphate group. LPA is not a single molecular entity but a collection of endogenous structural variants with fatty acids of varied lengths and degrees of saturation. Thus, when used herein, “LPA” refers to the set of bioactive LPA variants, unless stated otherwise. Biologically relevant variants of LPA include 18:2, 18:1, 18:0, 16:0 and 20:4. LPA species with both saturated fatty acids (16:0 and 18:0) and unsaturated fatty acids (16:1, 18:1, 18:2, and 20:4) have been detected in serum and plasma. The 16:0, 18:1, 18:2 and 20:4 LPA isoforms are the predominant species in blood. Significant levels (>1 μM) of bioactive LPA are detectable in various body fluids, including serum, saliva, follicular fluid and malignant effusions.

The present invention provides among its aspects anti-LPA agents that are useful for treating or preventing hyperproliferative disorders and various other disorders, as described in greater detail below. In particular, certain embodiments of the invention is drawn to antibodies targeted to LPA including but not limited to 18:2, 18:1, 18:0, 16:0, and 20:4 variants of LPA.

LPA has long been known as precursors of phospholipid biosynthesis in both eukaryotic and prokaryotic cells, but LPA has emerged only recently as a signaling molecule that are rapidly produced and released by activated cells, notably platelets, to influence target cells by acting on specific cell-surface receptor. Besides being synthesized and processed to more complex phospholipids in the endoplasmic reticulum, LPA can be generated through the hydrolysis of pre-existing phospholipids following cell activation; for example, the sn-2 position is commonly missing a fatty acid residue due to de-acylation, leaving only the sn-3 hydroxyl esterified to a fatty acid. Moreover, a key enzyme in the production of LPA, autotaxin (lysoPLD/NPP2), may be the product of an oncogene, as many tumor types up-regulate autotoxin. The concentrations of LPA in human plasma and serum have been reported, including determinations made using sensitive and specific LC/MS procedures. For example, in freshly prepared human serum allowed to sit at 25° C. for one hour, LPA concentrations have been estimated to be approximately 1.2 mM, with the LPA analogs 16:0, 18:1, 18:2, and 20:4 being the predominant species. Similarly, in freshly prepared human plasma allowed to sit at 25° C. for one hour, LPA concentrations have been estimated to be approximately 0.7 mM, with 18:1 and 18:2 LPA being the predominant species.

LPA mediates its biological functions predominantly by binding to a class of multiple transmembrane G protein-coupled receptors (GPCR). Five LPA-specific GPCRs, termed LPA1-5, have been identified to date; they show both overlapping and distinct signaling properties and tissue expression. The LPA1-3 receptors belong to the so-called EDG subfamily (EGD2/LPA1, EDG4/LPA2, and EDG7/LPA3) of GPCRs with 50% sequence similarity to each other. Their closest relative is the cannabinoid CB 1 receptor, which binds the bioactive lipids 2-arachidonoyl-glycerol (2-AG) and arachidonoyl-ethanolamine. Two newly identified LPA receptors, termed LPA4 (formerly GPR23/p2y9) and LPA5 (formerly GPR92) are more closely related to the P2Y nucleotide receptors. In addition, LPA recognizes the intracellular receptor, PPRgamma.

LPA1 is expressed in a wide range of tissues and organs whereas LPA2 and LPA3 show more restricted expression profile. However, LPA2 and LPA3 expressions were shown to be increased in ovarian and colon cancers and inflammation, suggesting that the main role of LPA2 and LPA3 is in pathophysiological conditions.

The role of these receptors has been in part elucidated by receptor knockout studies in mice. LPA1-deficient mice show partial postnatal lethality due to a suckling defect resulting from impaired olfaction. LPA1-deficient mice are also protected from lung fibrosis in response to bleomycin-induced lung injury. Furthermore, mice lacking the LPA1 receptor gene lose the nerve injury-induced neuropathic pain behaviors and phenomena.

In contrast, mice lacking LPA2 receptors appear to be normal. LPA3 receptor knockout mice have reduced litter size due to delayed blastocyst implantation and altered embryo spacing, and LPA3-deficient uteri show reduced cyclooxygenase-2 (COX-2) expression and prostaglandin synthesis; while exogenous administration of PGE2 into LPA3-deficient female mice has been reported to rescue the implantation defect.

LPAs influence a wide range of biological responses, including induction of cell proliferation, stimulation of cell migration and neurite retraction, gap junction closure, and even slime mold chemotaxis. The body of knowledge about the biology of LPA continues to grow as more and more cellular systems are tested for LPA responsiveness. The major physiological and pathophysiological effects of LPA include, for example:

Wound healing: It is now known that, in addition to stimulating cell growth and proliferation, LPA promote cellular tension and cell-surface fibronectin binding, which are important events in wound repair and regeneration.

Apoptosis: Recently, anti-apoptotic activity has also been ascribed to LPA, and it has recently been reported that peroxisome proliferation receptor gamma is a receptor/target for LPA.

Blood vessel maturation: Autotaxin, a secreted lysophospholipase D responsible for producing LPAs, is essential for blood vessel formation during development. In addition, unsaturated LPAs were identified as major contributors to the induction of vascular smooth muscle cell dedifferentiation.

Edema and vascular permeability: LPA induces plasma exudation and histamine release in mice.

Inflammation: LPA acts as inflammatory mediator in human corneal epithelial cells. LPA participates in corneal wound healing and stimulates the release of ROS in lens. LPA can also re-activate HSV-1 in rabbit cornea.

The bite of the venomous spider, Loxosceles reclusa (brown recluse spider), causes necrotic ulcers that can cause serious and long lasting tissue damage, and occasionally death.

The pathology of wounds generated from the bite of this spider consists of an intense inflammatory response mediated by AA and prostaglandins. The major component of the L. reclusa spider venom is the phospholipase D enzyme often referred to as sphingomyelinase D (SMase D), which hydrolyzes sphingomyelin to produce C1P. It has been found, however, that lysophospholipids with a variety of headgroups are hydrolysed by the L. reclusa enzyme to release LPA. It is believed that anti-LPA agents such as those of the invention will be useful in reducing or treating inflammation of various types, including but not limited to inflammation resulting from L. reclusa envenomation.

Fibrosis and scar formation: LPA inhibits TGF-mediated stimulation of type I collagen mRNA stability via an ERK-dependent pathway in dermal fibroblasts. Moreover, LPA have some direct fibrogenic effects by stimulating collagen gene expression and proliferation of fibroblasts.

Immune response: LPA, like S1P, has been shown to play a role in the immune response through modulation of immune-related cells. These lipids promote T-cell migration to sites of immune response and regulate proliferation of T cells as well as secretion of various cytokines.

Thus agents that reduce the effective concentration of LPA, such as Lpath's anti-LPA monoclonal antibodies, are believed to be useful in methods for treating diseases and conditions such as those associated with wound healing and fibrosis, apoptosis, angiogenesis and neovascularization, vascular permeability and inflammation, that are associated with an undesired, excessive or aberrant level of LPA.

Although polyclonal antibodies against naturally-occurring LPA have been reported in the literature (Chen, et al. (2000), Bioorg Med Chem Lett., August 7; 10(15):1691-3), monoclonal antibodies had not been described until the applicants developed several monoclonal antibodies, including humanized monoclonal antibodies, against LPAs. For example, see U.S. Patent Application Publication No. 20100034814, which is commonly owned with the instant application and is incorporated herein in its entirety. These anti-LPA antibodies can neutralize various LPAs and mitigate their biologic and pharmacologic action. Anti-LPA antibodies are, therefore, believed to be useful in prevention and/or treatment of various diseases and conditions associated with excessive, unwanted or aberrant levels of LPA.

Rapid and specific methods of detecting LPA are also desired. Methods for separating and semi-quantitatively measuring phospholipids such as LPA using techniques such as thin-layer chromatography (TLC) followed by gas chromatography (GC) and/or mass spectrometry (MS) are known. For example, lipids may be extracted from the test sample of bodily fluid. Alternatively, thin-layer chromatography may be used to separate various phospholipids. Phospholipids and lysophospholipids can then be visualized on plates, for example, using ultraviolet light. Alternatively, lysophospholipid concentrations can be identified by NMR or HPLC following isolation from phospholipids or as part of the phospholipid. LPA levels have also been determined in ascites from ovarian cancer patients using an assay that relies on LysoPA-specific effects on eukaryotic cells in culture. However, these prior procedures are time-consuming, expensive and variable and typically only semi-quantitative. Enzymatic methods for detecting lysophospholipids such as LPA in biological fluids, and for correlating and detecting conditions associated with altered levels of lysophospholipids, are also known. U.S. Pat. Nos. 6,255,063 and 6,248,553, originally assigned to Atairgin Technologies, Inc. and now commonly owned with the instant invention. The antibodies disclosed herein provide the basis for sensitive and specific methods for detection of LPA.

### 3. Definitions

Before describing the instant invention in detail, several terms used in the context of the present invention will be defined. In addition to these terms, others are defined elsewhere in the specification, as necessary. Unless otherwise expressly defined herein, terms of art used in this specification will have their art-recognized meanings.

The term “aberrant” means excessive or unwanted, for example in reference to levels or effective concentrations of a cellular target such as a protein or bioactive lipid.

The term “antibody” (“Ab”) or “immunoglobulin” (Ig) refers to any form of a peptide, polypeptide derived from, modeled after or encoded by, an immunoglobulin gene, or fragment thereof, that is capable of binding an antigen or epitope. See, e.g., Immunobiology, Fifth Edition, C. A. Janeway, P. Travers, M., Walport, M. J. Shlomchiked., ed. Garland Publishing (2001). The term “antibody” is used herein in the broadest sense, and encompasses monoclonal, polyclonal or multispecific antibodies, minibodies, heteroconjugates, diabodies, triabodies, chimeric, antibodies, synthetic antibodies, antibody fragments, and binding agents that employ the complementarity determining regions (CDRs) (or variants thereof that retain antigen binding activity) of the parent antibody. Antibodies are defined herein as retaining at least one desired activity of the parent antibody. Desired activities can include the ability to bind the antigen specifically, the ability to inhibit proleration in vitro, the ability to inhibit angiogenesis in vivo, and the ability to alter cytokine profile(s) in vitro. Herein, antibodies and antibody fragments, variants, and derivatives may also be referred to as “immune-derived moieties”, in that such molecules, or at least the antigen-binding portion(s) thereof, have been derived from an anti-LPA antibody.

Native antibodies (native immunoglobulins) are usually heterotetrameric glycoproteins of about 150,000 Daltons, typically composed of two identical light (L) chains and two identical heavy (H) chains. Each light chain is typically linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH), also referred to as the variable domain, followed by a number of constant domains. Each light chain has a variable domain at one end (VL) and a constant domain at its other end; the constant domain of the light chain is aligned with the first constant domain of the heavy chain, and the light-chain variable domain is aligned with the variable domain of the heavy chain. Particular amino acid residues form an interface between the light- and heavy-chain variable domains. The terms “variable domain” and “variable region” are used interchangeably. The terms “constant domain” and “constant region” are also interchangeable with each other.

Three hypervariable regions (also known as complementarity determining regions or CDRs) in each of the VH and VL regions form the unique antigen binding site of the molecule. Most of the amino acid sequence variation in the antibody molecule is within the CDRs, giving the antibody its specificity for its antigen.

The light chains of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

An “antibody derivative” is an immune-derived moiety, i.e., a molecule that is derived from an antibody. This comprehends, for example, antibody variants, antibody fragments, chimeric antibodies, humanized antibodies, multivalent antibodies, antibody conjugates and the like, which retain a desired level of binding activity for antigen.

As used herein, “antibody fragment” refers to a portion of an intact antibody that includes the antigen binding site or variable domains of an intact antibody, wherein the portion can be free of the constant heavy chain domains (e.g., CH2, CH3, and CH4) of the Fc region of the intact antibody. Alternatively, portions of the constant heavy chain domains (e.g., CH2, CH3, and CH4) can be included in the “antibody fragment”. Antibody fragments retain antigen-binding and include Fab, Fab′, F(ab′)2, Fd, and Fv fragments; diabodies; triabodies; single-chain antibody molecules (sc-Fv); minibodies, nanobodies, and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen. By way of example, a Fab fragment also contains the constant domain of a light chain and the first constant domain (CH1) of a heavy chain. “Fv” is the minimum antibody fragment that contains a complete antigen-recognition and -binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site. “Single-chain Fv” or “sFv” antibody fragments comprise the VH and VL domains of antibody, wherein these domains are present in a single polypeptide chain. Generally, the Fv polypeptide further comprises a polypeptide linker between the VH and VL domains that enables the sFv to form the desired structure for antigen binding. For a review of sFv, see Pluckthun in The Pharmacology of Monoclonal Antibodies, vol. 113, Rosenburg and Moore eds. Springer-Verlag, New York, pp. 269-315 (1994).

The Fab fragment also contains the constant domain of the light chain and the first constant domain (CH1) of the heavy chain. Fab′ fragments differ from Fab fragments by the addition of a few residues at the carboxyl terminus of the heavy chain CH1 domain including one or more cysteine(s) from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

An “antibody variant,” in this case an anti-LPA antibody variant, refers herein to a molecule which differs in amino acid sequence from a native anti-LPA antibody amino acid sequence by virtue of addition, deletion and/or substitution of one or more amino acid residue(s) in the antibody sequence and which retains at least one desired activity of the parent anti-binding antibody. Desired activities can include the ability to bind the antigen specifically, the ability to inhibit proliferation in vitro, the ability to inhibit angiogenesis in vivo, and the ability to alter cytokine profile in vitro. The amino acid change(s) in an antibody variant may be within a variable domain or a constant region of a light chain and/or a heavy chain, including in the Fc region, the Fab region, the CH1 domain, the CH2 domain, the CH3 domain, and the hinge region. In one embodiment, the variant comprises one or more amino acid substitution(s) in one or more hypervariable region(s) of the parent antibody. For example, the variant may comprise at least one, e.g. from about one to about ten, and preferably from about two to about five, substitutions in one or more hypervariable regions of the parent antibody. Ordinarily, the variant will have an amino acid sequence having at least 65% amino acid sequence identity with the parent antibody heavy or light chain variable domain sequences, more preferably at least 75%, more preferably at 80%, more preferably at least 85%, more preferably at least 90%, and most preferably at least 95%. In some situations a sequence identity of at least 50% is preferred, where other characteristics of the molecule convey desired attributes such as binding and specificity. Identity or homology with respect to this sequence is defined herein as the percentage of amino acid residues in the candidate sequence that are identical with the parent antibody residues, after aligning the sequences and introducing gaps, if necessary, to achieve the maximum percent sequence identity. None of N-terminal, C-terminal, or internal extensions, deletions, or insertions into the antibody sequence shall be construed as affecting sequence identity or homology. The variant retains the ability to bind LPA and preferably has desired activities which are superior to those of the parent antibody. For example, the variant may have a stronger binding affinity, enhanced ability to reduce angiogenesis and/or halt tumor progression. To analyze such desired properties (for example les immunogenic, longer half-life, enhanced stability, enhanced potency), one should compare a Fab form of the variant to a Fab form of the parent antibody or a full length form of the variant to a full length form of the parent antibody, for example, since it has been found that the format of the anti-sphingolipid antibody impacts its activity in the biological activity assays disclosed herein. The variant antibody of particular interest herein can be one which displays at least about 10 fold, preferably at least about % 5, 25, 59, or more of at least one desired activity. The preferred variant is one that has superior biophysical properties as measured in vitro or superior activities biological as measured in vitro or in vivo when compared to the parent antibody.

An “anti-LPA agent” refers to any therapeutic agent that binds LPA, and includes antibodies, antibody variants, antibody-derived molecules or non-antibody-derived moieties that bind LPA and its variants.

A “bioactive lipid” refers to a lipid signaling molecule. Bioactive lipids are distinguished from structural lipids (e.g., membrane-bound phospholipids) in that they mediate extracellular and/or intracellular signaling and thus are involved in controlling the function of many types of cells by modulating differentiation, migration, proliferation, secretion, survival, and other processes. In vivo, bioactive lipids can be found in extracellular fluids, where they can be complexed with other molecules, for example serum proteins such as albumin and lipoproteins, or in “free” form, i.e., not complexed with another molecule species. As extracellular mediators, some bioactive lipids alter cell signaling by activating membrane-bound ion channels or GPCRs or enzymes or factors that, in turn, activate complex signaling systems that result in changes in cell function or survival. As intracellular mediators, bioactive lipids can exert their actions by directly interacting with intracellular components such as enzymes, ion channels, or structural elements such as actin.

Examples of bioactive lipids include sphingolipids such as ceramide, ceramide-1-phosphate (C1P), sphingosine, sphinganine, sphingosylphosphorylcholine (SPC) and sphingosine-1-phosphate (S1P). Sphingolipids and their derivatives and metabolites are characterized by a sphingoid backbone (derived from sphingomyelin). Sphingolipids and their derivatives and metabolites represent a group of extracellular and intracellular signaling molecules with pleiotropic effects on important cellular processes. They include sulfatides, gangliosides and cerebrosides. Other bioactive lipids are characterized by a glycerol-based backbone; for example, lysophospholipids such as lysophosphatidyl choline (LPC) and various lysophosphatidic acids (LPA), as well as phosphatidylinositol (PI), phosphatidylethanolamine (PEA), phosphatidic acid, platelet activating factor (PAF), cardiolipin, phosphatidylglycerol (PG) and diacylglyceride (DG). Yet other bioactive lipids are derived from arachidonic acid; these include the eicosanoids (including the eicosanoid metabolites such as the HETEs, cannabinoids, leukotrienes, prostaglandins, lipoxins, epoxyeicosatrienoic acids, and isoeicosanoids), non-eicosanoid cannabinoid mediators. Other bioactive lipids, including other phospholipids and their derivatives, may also be used according to the instant invention.

In some embodiments of the invention it may be preferable to target glycerol-based bioactive lipids (those having a glycerol-derived backbone, such as the LPAs) for antibody production, as opposed to sphingosine-based bioactive lipids (those having a sphingoid backbone, such as sphingosine and S1P). In other embodiments it may be desired to target arachidonic acid-derived bioactive lipids for antibody generation, and in other embodiments arachidonic acid-derived and glycerol-derived bioactive lipids but not sphingoid-derived bioactive lipids are preferred. Together the arachidonic acid-derived and glycerol-derived bioactive lipids may be referred to herein as “non-sphingoid bioactive lipids.”

Specifically excluded from the class of bioactive lipids according to the invention are phosphatidylcholine and phosphatidylserine, as well as their metabolites and derivatives that function primarily as structural members of the inner and/or outer leaflet of cellular membranes.

The term “biologically active,” in the context of an antibody or antibody fragment or variant, refers to an antibody or antibody fragment or antibody variant that is capable of binding the desired epitope and in some ways exerting a biologic effect. Biological effects include, but are not limited to, the modulation of a growth signal, the modulation of an anti-apoptotic signal, the modulation of an apoptotic signal, the modulation of the effector function cascade, and modulation of other ligand interactions.

A “biomarker” is a specific biochemical in the body which has a particular molecular feature that makes it useful for measuring the progress of disease or the effects of treatment. For example, S1P is a biomarker for certain hyperproliferative and/or cardiovascular conditions.

“Cardiovascular therapy” encompasses cardiac therapy (treatment of myocardial ischemia and heart failure) as well as the prevention and/or treatment of other diseases associated with the cardiovascular system, such as heart disease. The term “heart disease” encompasses any type of disease, disorder, trauma, or surgical treatment that involves the heart or myocardial tissue. Of particular interest are conditions associated with tissue remodeling. The term “cardiotherapeutic agent” refers to an agent that is therapeutic to diseases and diseases caused by or associated with cardiac and myocardial diseases and disorders.

A “carrier” refers to a moiety adapted for conjugation to a hapten, thereby rendering the hapten immunogenic. A representative, non-limiting class of carriers is proteins, examples of which include albumin, keyhole limpet hemocyanin, hemaglutanin, tetanus, and diptheria toxoid. Other classes and examples of carriers suitable for use in accordance with the invention are known in the art. These, as well as later discovered or invented naturally occurring or synthetic carriers, can be adapted for application in accordance with the invention.

As used herein, the expressions “cell,” “cell line,” and “cell culture” are used interchangeably and all such designations include progeny. Thus, the words “transformants” and “transformed cells” include the primary subject cell and cultures derived there from without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

The term “chemotherapeutic agent” means anti-cancer and other anti-hyperproliferative agents. Thus chemotherapeutic agents are a subset of therapeutic agents in general. Chemotherapeutic agents include, but are not limited to: DNA damaging agents and agents that inhibit DNA synthesis: anthracyclines (doxorubicin, donorubicin, epirubicin), alkylating agents (bendamustine, busulfan, carboplatin, carmustine, chlorambucil, cyclophosphamide, dacarbazine, hexamethylmelamine, ifosphamide, lomustine, mechlorethamine, melphalan, mitotane, mytomycin, pipobroman, procarbazine, streptozocin, thiotepa, and triethylenemelamine), platinum derivatives (cisplatin, carboplatin, cis diammine-dichloroplatinum), and topoisomerase inhibitors (Camptosar); anti-metabolites such as capecitabine, chlorodeoxyadenosine, cytarabine (and its activated form, ara-CMP), cytosine arabinoside, dacabazine, floxuridine, fludarabine, 5-fluorouracil, 5-DFUR, gemcitabine, hydroxyurea, 6-mercaptopurine, methotrexate, pentostatin, trimetrexate, 6-thioguanine); anti-angiogenics (bevacizumab, thalidomide, sunitinib, lenalidomide, TNP-470, 2-methoxyestradiol, ranibizumab, sorafenib, erlotinib, bortezomib, pegaptanib, endostatin); vascular disrupting agents (flavonoids/flavones, DMXAA, combretastatin derivatives such as CA4DP, ZD6126, AVE8062A, etc.); biologics such as antibodies (Herceptin, Avastin, Panorex, Rituxin, Zevalin, Mylotarg, Campath, Bexxar, Erbitux); endocrine therapy: aromatase inhibitors (4-hydroandrostendione, exemestane, aminoglutehimide, anastrazole, letozole), anti-estrogens (Tamoxifen, Toremifine, Raoxifene, Faslodex), steroids such as dexamethasone; immuno-modulators: cytokines such as IFN-beta and IL2), inhibitors to integrins, other adhesion proteins and matrix metalloproteinases); histone deacetylase inhibitors like suberoylanilide hydroxamic acid; inhibitors of signal transduction such as inhibitors of tyrosine kinases like imatinib (Gleevec); inhibitors of heat shock proteins like 17-N-allylamino-17-demethoxygeldanamycin; retinoids such as all trans retinoic acid; inhibitors of growth factor receptors or the growth factors themselves; anti-mitotic compounds and/or tubulin-depolymerizing agents such as the taxoids (paclitaxel, docetaxel, taxotere, BAY 59-8862), navelbine, vinblastine, vincristine, vindesine and vinorelbine; anti-inflammatories such as COX inhibitors and cell cycle regulators, e.g., check point regulators and telomerase inhibitors.

The term “chimeric” antibody (or immunoglobulin) refers to a molecule comprising a heavy and/or light chain which is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (Cabilly, et al., infra; Morrison et al., Proc. Natl. Acad. Sci. U.S.A., vol. 81:6851 (1984)). One example of a chimeric antibody is an antibody containing murine variable domains (VL and VH) and human constant domains. However, antibody sequences may be vertebrate or invertebrate in origin, e.g., from mammal, bird or fish, including cartilaginous fish, rodents, canines, felines, ungulate animals and primates, including humans.

The term “combination therapy” refers to a therapeutic regimen that involves the provision of at least two distinct therapies to achieve an indicated therapeutic effect. For example, a combination therapy may involve the administration of two or more chemically distinct active ingredients, for example, a fast-acting chemotherapeutic agent and an anti-lipid antibody. Alternatively, a combination therapy may involve the administration of an anti-lipid antibody and/or one or more chemotherapeutic agents, alone or together with the delivery of another treatment, such as radiation therapy and/or surgery. In the context of the administration of two or more chemically distinct active ingredients, it is understood that the active ingredients may be administered as part of the same composition or as different compositions. When administered as separate compositions, the compositions comprising the different active ingredients may be administered at the same or different times, by the same or different routes, using the same of different dosing regimens, all as the particular context requires and as determined by the attending physician. Similarly, when one or more anti-lipid antibody species, for example, an anti-LPA antibody, alone or in conjunction with one or more chemotherapeutic agents are combined with, for example, radiation and/or surgery, the drug(s) may be delivered before or after surgery or radiation treatment.

The expression “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

A “derivatized bioactive lipid” is a bioactive lipid, e.g., LPA, which has a polar head group and at least one hydrocarbon chain, wherein a carbon atom within the hydrocarbon chain is derivatized with a pendant reactive group (e.g., a sulfhydryl (thiol) group, a carboxylic acid group, a cyano group, an ester, a hydroxy group, an alkene, an alkyne, an acid chloride group or a halogen atom) that may or may not be protected. This derivatization serves to activate the bioactive lipid for reaction with a molecule, e.g., for conjugation to a carrier.

A “derivatized bioactive lipid conjugate” refers to a derivatized bioactive lipid that is covalently conjugated to a carrier. The carrier may be a protein molecule or may be a moiety such as polyethylene glycol, colloidal gold, adjuvants or silicone beads. A derivatized bioactive lipid conjugate may be used as an immunogen for generating an antibody response according to the instant invention, and the same or a different bioactive lipid conjugate may be used as a detection reagent for detecting the antibody thus produced. In some embodiments the derivatized bioactive lipid conjugate is attached to a solid support when used for detection.

The term “diabodies” refers to small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger, et al., Proc. Natl. Acad. Sci. USA 90:6444-6448 (1993).

“Effective concentration” refers to the absolute, relative, and/or available concentration and/or activity, for example of certain undesired bioactive lipids. In other words, the effective concentration of a bioactive lipid is the amount of lipid available, and able, to perform its biological function. In the present invention, an immune-derived moiety such as, for example, a monoclonal antibody directed to a bioactive lipid (such as, for example, C1P) is able to reduce the effective concentration of the lipid by binding to the lipid and rendering it unable to perform its biological function. In this example, the lipid itself is still present (it is not degraded by the antibody, in other words) but can no longer bind its receptor or other targets to cause a downstream effect, so “effective concentration” rather than absolute concentration is the appropriate measurement. Methods and assays exist for directly and/or indirectly measuring effective concentrations of bioactive lipids.

An “epitope” or “antigenic determinant” refers to that portion of an antigen that reacts with an antibody antigen-binding portion derived from an antibody.

The term “expression cassette” refers to a nucleotide molecule capable of affecting expression of a structural gene (i.e., a protein coding sequence, such as an antibody of the invention) in a host compatible with such sequences. Expression cassettes include at least a promoter operably linked with the polypeptide-coding sequence, and, optionally, with other sequences, e.g., transcription termination signals. Additional regulatory elements necessary or helpful in effecting expression may also be used, e.g., enhancers. Thus, expression cassettes include plasmids, expression vectors, recombinant viruses, any form of recombinant “naked DNA” vector, and the like.

A “fully human antibody” can refer to an antibody produced in a genetically engineered (i.e., transgenic) animal, typically a mammal, usually a mouse (e.g., as can be obtained from Medarex) that, when presented with a suitable immunogen, can produce a human antibody that does not necessarily require CDR grafting. These antibodies are fully “human” in that they generated from from an animal (e.g., a transgenic mouse) in which the non-human antibody genes are replaced or suppressed and replaced with some or all of the human immunoglobulin genes. In other words, antibodies of the invention include those generated against bioactive lipids, specifically LPA, when presented in an immunogenic form to mice or other animals genetically engineered to produce human frameworks for relevant CDRs.

A “hapten” is a substance that is non-immunogenic but can react with an antibody or antigen-binding portion derived from an antibody. In other words, haptens have the property of antigenicity but not immunogenicity. A hapten is generally a small molecule that can, under most circumstances, elicit an immune response (i.e., act as an antigen) only when attached to a carrier, for example, a protein, polyethylene glycol (PEG), colloidal gold, silicone beads, or the like. The carrier may be one that also does not elicit an immune response by itself.

The term “heteroconjugate antibody” can refer to two covalently joined antibodies. Such antibodies can be prepared using known methods in synthetic protein chemistry, including using crosslinking agents. As used herein, the term “conjugate” refers to molecules formed by the covalent attachment of one or more antibody fragment(s) or binding moieties to one or more polymer molecule(s).

“Humanized” forms of non-human (e.g., murine) antibodies are chimeric antibodies that contain minimal sequence derived from non-human immunoglobulin. Or, looked at another way, a humanized antibody is a human antibody that also contains selected sequences from non-human (e.g., murine) antibodies in place of the human sequences. A humanized antibody can include conservative amino acid substitutions or non-natural residues from the same or different species that do not significantly alter its binding and/or biologic activity. Such antibodies are chimeric antibodies that contain minimal sequence derived from non-human immunoglobulins. For the most part, humanized antibodies are human immunoglobulins (recipient antibody) in which residues from a complementary-determining region (CDR) of the recipient are replaced by residues from a CDR of a non-human species (donor antibody) such as mouse, rat, camel, bovine, goat, or rabbit having the desired properties. In some instances, framework region (FR) residues of the human immunoglobulin are replaced by corresponding non-human residues.

Furthermore, humanized antibodies can comprise residues that are found neither in the recipient antibody nor in the imported CDR or framework sequences. These modifications are made to further refine and maximize antibody performance. Thus, in general, a humanized antibody will comprise all of at least one, and in one aspect two, variable domains, in which all or all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), or that of a human immunoglobulin. See, e.g., Cabilly, et al., U.S. Pat. No. 4,816,567; Cabilly, et al., European Patent No. 0,125,023 B1; Boss, et al., U.S. Pat. No. 4,816,397; Boss, et al., European Patent No. 0,120,694 B1; Neuberger, et al., WO 86/01533; Neuberger, et al., European Patent No. 0,194,276 B1; Winter, U.S. Pat. No. 5,225,539; Winter, European Patent No. 0,239,400 B1; Padlan, et al., European Patent Application No. 0,519,596 A1; Queen, et al. (1989), Proc. Nat'l Acad. Sci. USA, vol. 86:10029-10033). For further details, see Jones, et al., Nature 321:522-525 (1986); Reichmann, et al., Nature 332:323-329 (1988); and Presta, Curr. Op. Struct. Biol. 2:593-596 (1992), and Hansen, WO2006105062.

The term “hyperproliferative disorder” refers to diseases and disorders associated with, the uncontrolled proliferation of cells, including but not limited to uncontrolled growth of organ and tissue cells resulting in cancers and benign tumors. Hyperproliferative disorders associated with endothelial cells can result in diseases of angiogenesis such as angiomas, endometriosis, obesity, age-related macular degeneration and various retinopathies, as well as the proliferation of endothelial cells and smooth muscle cells that cause restenosis as a consequence of stenting in the treatment of atherosclerosis. Hyperproliferative disorders involving fibroblasts (i.e., fibrogenesis) include, without limitation, disorders of excessive scarring (i.e., fibrosis) such as age-related macular degeneration, cardiac remodeling and failure associated with myocardial infarction, as well asexcessive wound healing such as commonly occurs as a consequence of surgery or injury, keloids, and fibroid tumors and stenting.

An “immunogen” is a molecule capable of inducing a specific immune response, particularly an antibody response in an animal to whom the immunogen has been administered. In the instant invention, the immunogen is a derivatized bioactive lipid conjugated to a carrier, i.e., a “derivatized bioactive lipid conjugate”. The derivatized bioactive lipid conjugate used as the immunogen may be used as capture material for detection of the antibody generated in response to the immunogen. Thus the immunogen may also be used as a detection reagent. Alternatively, the derivatized bioactive lipid conjugate used as capture material may have a different linker and/or carrier moiety from that in the immunogen.

To “inhibit,” particularly in the context of a biological phenomenon, means to decrease, suppress or delay. For example, a treatment yielding “inhibition of tumorigenesis” may mean that tumors do not form at all, or that they form more slowly, or are fewer in number than in the untreated control.

An “isolated” composition is one that has been identified and separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the composition is an antibody and will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The word “label” when used herein refers to a detectable compound or composition, such as one that is conjugated directly or indirectly to the antibody. The label may itself be detectable by itself (e.g., radioisotope labels or fluorescent labels) or, in the case of an enzymatic label, may catalyze chemical alteration of a substrate compound or composition that is detectable.

A “liposome” is a small vesicle composed of various types of lipids, phospholipids and/or surfactant that is useful for delivery of a drug (such as the anti-sphingolipid antibodies disclosed herein and, optionally, a chemotherapeutic agent) to a mammal. The components of the liposome are commonly arranged in a bilayer formation, similar to the lipid arrangement of biological membranes. An “isolated” nucleic acid molecule is a nucleic acid molecule that is identified and separated from at least one contaminant nucleic acid molecule with which it is ordinarily associated in the natural source of the antibody nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the antibody where, for example, the nucleic acid molecule is in a chromosomal location different from that of non-engineered cells.

In the context of this invention, a “liquid composition” refers to one that, in its filled and finished form as provided from a manufacturer to an end user (e.g., a doctor or nurse), is a liquid or solution, as opposed to a solid. Here, “solid” refers to compositions that are not liquids or solutions. For example, solids include dried compositions prepared by lyophilization, freeze-drying, precipitation, and similar procedures.

The expression “linear antibodies” when used throughout this application refers to the antibodies described in Zapata, et al. Protein Eng. 8(10):1057-1062 (1995). Briefly, these antibodies comprise a pair of tandem Fd segments (VH-CH1-VH-CH1) that form a pair of antigen binding regions. Linear antibodies can be bispecific or monospecific.

The term “metabolites” refers to compounds from which LPAs are made, as well as those that result from the degradation of LPAs; that is, compounds that are involved in the lysophospholipid metabolic pathways. The term “metabolic precursors” may be used to refer to compounds from which sphingolipids are made.

The term “monoclonal antibody” (mAb) as used herein refers to an antibody obtained from a population of substantially homogeneous antibodies, or to said population of antibodies. The individual antibodies comprising the population are essentially identical, except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler, et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson, et al., Nature 352:624-628 (1991) and Marks, et al., J. Mol. Biol. 222:581-597 (1991), for example, or by other methods known in the art. The monoclonal antibodies herein specifically include chimeric antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison, et al., Proc. Natl. Acad. Sci. USA 81:6851-6855 (1984)).

“Monotherapy” refers to a treatment regimen based on the delivery of one therapeutically effective compound, whether administered as a single dose or several doses over time.

The term “multispecific antibody” can refer to an antibody, or a monoclonal antibody, having binding properties for at least two different epitopes. In one embodiment, the epitopes are from the same antigen. In another embodiment, the epitopes are from two or more different antigens. Methods for making multispecific antibodies are known in the art. Multispecific antibodies include bispecific antibodies (having binding properties for two epitopes), trispecific antibodies (three epitopes) and so on. For example, multispecific antibodies can be produced recombinantly using the co-expression of two or more immunoglobulin heavy chain/light chain pairs. Alternatively, multispecific antibodies can be prepared using chemical linkage. One of skill can produce multispecific antibodies using these or other methods as may be known in the art. Multispecific antibodies include multispecific antibody fragments. One example of a multispecific (in this case, bispecific) antibody comprehended by this invention is an antibody having binding properties for an S1P epitope and a C1P epitope, which thus is able to recognize and bind to both S1P and C1P. Another example of of a bispecific antibody comprehended by this invention is an antibody having binding properties for an epitope from a bioactive lipid and an epitope from a cell surface antigen. Thus the antibody is able to recognize and bind the bioactive lipid and is able to recognize and bind to cells, e.g., for targeting purposes.

“Neoplasia” or “cancer” refers to abnormal and uncontrolled cell growth. A “neoplasm”, or tumor or cancer, is an abnormal, unregulated, and disorganized proliferation of cell growth, and is generally referred to as cancer. A neoplasm may be benign or malignant. A neoplasm is malignant, or cancerous, if it has properties of destructive growth, invasiveness, and metastasis. Invasiveness refers to the local spread of a neoplasm by infiltration or destruction of surrounding tissue, typically breaking through the basal laminas that define the boundaries of the tissues, thereby often entering the body's circulatory system. Metastasis typically refers to the dissemination of tumor cells by lymphatics or blood vessels. Metastasis also refers to the migration of tumor cells by direct extension through serous cavities, or subarachnoid or other spaces. Through the process of metastasis, tumor cell migration to other areas of the body establishes neoplasms in areas away from the site of initial appearance.

“Neuropathic pain” is the chronic pain state caused by pathologic changes in the nervous system.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The “parent” antibody herein is one that is encoded by an amino acid sequence used for the preparation of the variant. The parent antibody may be a native antibody or may already be a variant, e.g., a chimeric antibody. For example, the parent antibody may be a humanized or human antibody.

A “patentable” composition, process, machine, or article of manufacture according to the invention means that the subject matter satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically exclude the non-patentable embodiment(s). Also, the claims appended hereto are to be interpreted both to provide the broadest reasonable scope, as well as to preserve their validity. Furthermore, the claims are to be interpreted in a way that (1) preserves their validity and (2) provides the broadest reasonable interpretation under the circumstances, if one or more of the statutory requirements for patentability are amended or if the standards change for assessing whether a particular statutory requirement for patentability is satisfied from the time this application is filed or issues as a patent to a time the validity of one or more of the appended claims is questioned.

The term “pharmaceutically acceptable salt” refers to a salt, such as used in formulation, which retains the biological effectiveness and properties of the agents and compounds of this invention and which are is biologically or otherwise undesirable. In many cases, the agents and compounds of this invention are capable of forming acid and/or base salts by virtue of the presence of charged groups, for example, charged amino and/or carboxyl groups or groups similar thereto. Pharmaceutically acceptable acid addition salts may be prepared from inorganic and organic acids, while pharmaceutically acceptable base addition salts can be prepared from inorganic and organic bases. For a review of pharmaceutically acceptable salts (see Berge, et al. (1977), J. Pharm. Sci., vol. 66, 1-19).

A “plurality” means more than one.

The term “promoter” includes all sequences capable of driving transcription of a coding sequence in a cell. Thus, promoters used in the constructs of the invention include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. Transcriptional regulatory regions suitable for use in the present invention include but are not limited to the human cytomegalovirus (CMV) immediate-early enhancer/promoter, the SV40 early enhancer/promoter, the E. coli lac or trp promoters, and other promoters known to control expression of genes in prokaryotic or eukaryotic cells or their viruses.

The term “recombinant DNA” refers to nucleic acids and gene products expressed therefrom that have been engineered, created, or modified by man “Recombinant” polypeptides or proteins are polypeptides or proteins produced by recombinant DNA techniques, for example, from cells transformed by an exogenous DNA construct encoding the desired polypeptide or protein. “Synthetic” polypeptides or proteins are those prepared by chemical synthesis.

The terms “separated”, “purified”, “isolated”, and the like mean that one or more components of a sample contained in a sample-holding vessel are or have been physically removed from, or diluted in the presence of, one or more other sample components present in the vessel. Sample components that may be removed or diluted during a separating or purifying step include, chemical reaction products, non-reacted chemicals, proteins, carbohydrates, lipids, and unbound molecules.

By “solid phase” is meant a non-aqueous matrix such as one to which the antibody of the present invention can adhere. Examples of solid phases encompassed herein include those formed partially or entirely of glass (e.g. controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g. an affinity chromatography column). This term also includes a discontinuous solid phase of discrete particles, such as those described in U.S. Pat. No. 4,275,149.

The term “species” is used herein in various contexts, e.g., a particular species of chemotherapeutic agent. In each context, the term refers to a population of chemically indistinct molecules of the sort referred in the particular context.

The term “specific” or “specificity” in the context of antibody-antigen interactions refers to the selective, non-random interaction between an antibody and its target epitope. Here, the term “antigen” refers to a molecule that is recognized and bound by an antibody molecule or other immune-derived moiety. The specific portion of an antigen that is bound by an antibody is termed the “epitope”. This interaction depends on the presence of structural, hydrophobic/hydrophilic, and/or electrostatic features that allow appropriate chemical or molecular interactions between the molecules. Thus an antibody is commonly said to “bind” (or “specifically bind”) or be “reactive with” (or “specifically reactive with), or, equivalently, “reactive against” (or “specifically reactive against”) the epitope of its target antigen. Antibodies are commonly described in the art as being “against” or “to” their antigens as shorthand for antibody binding to the antigen. Thus an “antibody that binds C1P,” an “antibody reactive against C1P,” an “antibody reactive with C1P,” an “antibody to C1P” and an “anti-C1P antibody” all have the same meaning in the art. Antibody molecules can be tested for specificity of binding by comparing binding to the desired antigen to binding to unrelated antigen or analogue antigen or antigen mixture under a given set of conditions. Preferably, an antibody according to the invention will lack significant binding to unrelated antigens, or even analogs of the target antigen.

Herein, “stable” refers to an interaction between two molecules (e.g., a peptide and a TLR molecule) that is sufficiently stable such that the molecules can be maintained for the desired purpose or manipulation. For example, a “stable” interaction between a peptide and a TLR molecule refers to one wherein the peptide becomes and remains associated with a TLR molecule for a period sufficient to achieve the desired effect.

A “subject” or “patient” refers to an animal in need of treatment that can be effected by molecules of the invention. Animals that can be treated in accordance with the invention include vertebrates, with mammals such as bovine, canine, equine, feline, ovine, porcine, and primate (including humans and non-human primates) animals being particularly preferred examples.

A “surrogate marker” refers to laboratory measurement of biological activity within the body that indirectly indicates the effect of treatment on disease state. Examples of surrogate markers for hyperproliferative and/or cardiovascular conditions include SPHK and/or S1PRs.

A “therapeutic agent” refers to a drug or compound that is intended to provide a therapeutic effect including, but not limited to: anti-inflammatory drugs including COX inhibitors and other NSAIDS, anti-angiogenic drugs, chemotherapeutic drugs as defined above, cardiovascular agents, immunomodulatory agents, agents that are used to treat neurodegenerative disorders, opthalmic drugs, etc.

A “therapeutically effective amount” (or “effective amount”) refers to an amount of an active ingredient, e.g., an agent according to the invention, sufficient to effect treatment when administered to a subject in need of such treatment. Accordingly, what constitutes a therapeutically effective amount of a composition according to the invention may be readily determined by one of ordinary skill in the art. In the context of cancer therapy, a “therapeutically effective amount” is one that produces an objectively measured change in one or more parameters associated with cancer cell survival or metabolism, including an increase or decrease in the expression of one or more genes correlated with the particular cancer, reduction in tumor burden, cancer cell lysis, the detection of one or more cancer cell death markers in a biological sample (e.g., a biopsy and an aliquot of a bodily fluid such as whole blood, plasma, serum, urine, etc.), induction of induction apoptosis or other cell death pathways, etc. Of course, the therapeutically effective amount will vary depending upon the particular subject and condition being treated, the weight and age of the subject, the severity of the disease condition, the particular compound chosen, the dosing regimen to be followed, timing of administration, the manner of administration and the like, all of which can readily be determined by one of ordinary skill in the art. It will be appreciated that in the context of combination therapy, what constitutes a therapeutically effective amount of a particular active ingredient may differ from what constitutes a therapeutically effective amount of the active ingredient when administered as a monotherapy (i.e., a therapeutic regimen that employs only one chemical entity as the active ingredient).

The compositions of the invention are used in methods of bioactive lipid-based therapy. As used herein, the terms “therapy” and “therapeutic” encompasses the full spectrum of prevention and/or treatments for a disease, disorder or physical trauma. A “therapeutic” agent of the invention may act in a manner that is prophylactic or preventive, including those that incorporate procedures designed to target individuals that can be identified as being at risk (pharmacogenetics); or in a manner that is ameliorative or curative in nature; or may act to slow the rate or extent of the progression of at least one symptom of a disease or disorder being treated; or may act to minimize the time required, the occurrence or extent of any discomfort or pain, or physical limitations associated with recuperation from a disease, disorder or physical trauma; or may be used as an adjuvant to other therapies and treatments.

The term “treatment” or “treating” means any treatment of a disease or disorder, including preventing or protecting against the disease or disorder (that is, causing the clinical symptoms not to develop); inhibiting the disease or disorder (i.e., arresting, delaying or suppressing the development of clinical symptoms; and/or relieving the disease or disorder (i.e., causing the regression of clinical symptoms). As will be appreciated, it is not always possible to distinguish between “preventing” and “suppressing” a disease or disorder because the ultimate inductive event or events may be unknown or latent. Those “in need of treatment” include those already with the disorder as well as those in which the disorder is to be prevented. Accordingly, the term “prophylaxis” will be understood to constitute a type of “treatment” that encompasses both “preventing” and “suppressing”. The term “protection” thus includes “prophylaxis”.

The term “therapeutic regimen” means any treatment of a disease or disorder using chemotherapeutic and cytotoxic agents, radiation therapy, surgery, gene therapy, DNA vaccines and therapy, siRNA therapy, anti-angiogenic therapy, immunotherapy, bone marrow transplants, aptamers and other biologics such as antibodies and antibody variants, receptor decoys and other protein-based therapeutics.

The “variable” region of an antibody comprises framework and complementarity determining regions (CDRs, otherwise known as hypervariable regions). The variability is not evenly distributed throughout the variable domains of antibodies. It is concentrated in six CDR segments, three in each of the light chain and the heavy chain variable domains. The more highly conserved portions of variable domains are called the framework region (FR). The variable domains of native heavy and light chains each comprise four FRs (FR1, FR2, FR3 and FR4, respectively), largely adopting a β-sheet configuration, connected by three hypervariable regions, which form loops connecting, and in some cases forming part of, the beta-sheet structure. The term “hypervariable region” when used herein refers to the amino acid residues of an antibody which are responsible for antigen binding. The hypervariable region comprises amino acid residues from a “complementarity determining region” or “CDR” (for example residues 24-34 (L1), 50-56 (L2) and 89-97 (L3) in the light chain variable domain and 31-35 (H1), 50-65 (H2) and 95-102 (H3) in the heavy chain variable domain; Kabat, et al., Sequences of Proteins of Immunological Interest, 5th Ed. Public Health Service, National Institutes of Health, Bethesda, Md. (1991)) and/or those residues from a “hypervariable loop” (for example, residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework” or “FR” residues are those variable domain residues other than the hypervariable region residues as herein defined.

The hypervariable regions in each chain are held together in close proximity by the FRs and, with the hypervariable regions from the other chain, contribute to the formation of the antigen-binding site of antibodies (see Kabat, et al., Sequences of Proteins of Immunological Interest, 5th Ed. Public Health Service, National Institutes of Health, Bethesda, Md. (1991), pages 647-669). The constant domains are not involved directly in binding an antibody to an antigen, but exhibit various effector functions, such as participation of the antibody in antibody-dependent cellular toxicity.

A “vector” or “plasmid” or “expression vector” refers to a nucleic acid that can be maintained transiently or stably in a cell to effect expression of one or more recombinant genes. A vector can comprise nucleic acid, alone or complexed with other compounds. A vector optionally comprises viral or bacterial nucleic acids and/or proteins, and/or membranes. Vectors include, but are not limited, to replicons (e.g., RNA replicons, bacteriophages) to which fragments of DNA may be attached and become replicated. Thus, vectors include, but are not limited to, RNA, autonomous self-replicating circular or linear DNA or RNA and include both the expression and non-expression plasmids. Plasmids can be commercially available, publicly available on an unrestricted basis, or can be constructed from available plasmids as reported with published protocols. In addition, the expression vectors may also contain a gene to provide a phenotypic trait for selection of transformed host cells such as dihydrofolate reductase or neomycin resistance for eukaryotic cell culture, or such as tetracycline or ampicillin resistance in E. coli.

### SUMMARY OF THE INVENTION

The present application discloses methods of treating or preventing pain associated with aberrant levels of LPA, which methods comprise administering to a subject, including a human subject, having or believed to be at risk of having pain associated with aberrant levels of LPA an antibody that binds LPA, in an amount effective to reduce in vivo the effective concentration of LPA. The antibody may be a polyclonal or monoclonal antibody, or a fragment of these which retains binding ability for LPA. The pain may be acute or chronic neuropathic pain and/or may be, e.g., due to injury, trauma, or damage to the central or peripheral nervous system, inflammation, drug exposure, diabetes, viral disease, metabolic disease, ischemic insult, nutrient deficiency, toxin exposure, cancer, or cancer treatment.

The foregoing and other aspects of the invention will become more apparent from the following detailed description, accompanying drawings, and the claims. Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### BRIEF DESCRIPTION OF THE DRAWINGS

This application contains at least one figure executed in color. Copies of this application with color drawing(s) will be provided upon request and payment of the necessary fee. A brief summary of each of the figures is provided below.

FIGS. 1A-1B. FIG. 1A is a time line and bar graph showing the effect of prophylactic anti-LPA antibody treatment on paw withdrawal latency (PWL), a measure of pain. FIG. 1B is a time line and bar graph showing the effect of interventional anti-LPA antibody treatment on PWL. Both the prophylactic and interventional treatments decreased pain in this model.

FIG. 2 is a bar graph showing inhibition of pain vocalization in arthritic rats after treatment with humanized anti-LPA antibody LT3015. The antibody was given at three doses (1.6, 8 and 40 mg/kg) in this preliminary study. The two higher doses decreased pain vocalization to the level seen after treatment with Naproxen, the positive control. The lowest dose (1.6 mg/kg) had an intermediate effect and the nonspecific antibody had a minimal effect on pain vocalization.

### DETAILED DESCRIPTION OF THE INVENTION

### Anti-LPA Agents, Including Anti-LPA Antibodies

1. Introduction

The use of monoclonal antibodies (mAbs) as a therapeutic treatment for a variety of diseases and disorders is rapidly increasing because they have been shown to be safe and efficacious therapeutic agents. Approved therapeutic monoclonal antibodies include Avastin™, Erbitux™, and Rituxan™. Additional monoclonal antibodies are in various phases of clinical development for a variety of diseases with the majority targeting various forms of cancer. In general, monoclonal antibodies are generated in non-human mammals. The therapeutic utility of murine monoclonal antibodies may be improved with chimerization or humanization of non-human mammalian antibodies. Humanization greatly lessens the development of an immune response against the administered therapeutic monoclonal antibodies and thereby avoids the reduction of half-life and therapeutic efficacy consequent on such a response. For the most part, the humanization process consists of grafting the murine complementary determining regions (CDRs) into the framework region (FR) of a human immunoglobulin. Backmutation to murine amino acid residues of selected residues in the FR is often required to improve or regain affinity that is lost in the initial grafted construct.

The manufacture of monoclonal antibodies is a complex process that stems from the variability of the immunoglobulin protein itself. The heterogeneity can be attributed to the formation of alternative disulfide pairings, deamidation and the formation of isoaspartyl residues, methionine and cysteine oxidation, cyclization of N-terminal glutamine residues to pyroglutamate and partial enzymatic cleavage of C-terminal lysines by mammalian carboxypeptidases. Engineering is commonly applied to antibody molecules to improve their properties, such as enhanced stability, resistance to proteases, aggregation behavior and enhance the expression level in heterologous systems.

2. Disease Associations of LPA and Therapeutic Uses for Anti-LPA Agents

LPA has been associated with a number of diseases and disorders. For review, see Gardell, et al., (2006) Trends Mol Med. 12(2):65-75, and Chun J. and Rosen, H., (2006) Curr. Pharma. Design 12:161-171. These include autoimmune disorders such as diabetes, multiple sclerosis and scleroderma; hyperproliferative disorders including cancer; pain, disorders associated with angiogenesis and neovascularization; obesity; neurodegenerative diseases including Alzheimer's disease; schizophrenia, immune-related disorders such as transplant rejection and graft-vs.-host disease, and others. The roles of LPA in many diseases and disorders is further described in, for example, U.S. Patent Application Publication No. 20100034814, which is commonly owned with the instant application and is incorporated herein in its entirety and for all purposes.

a. Hyperproliferative Disorders

One aspect of the invention concerns methods for treating hyperproliferative disorders. These methods comprise administering to a mammal (e.g., a bovine, canine, equine, ovine, or porcine animal, particularly a human) known or suspected to suffer from an LPA-associated hyperproliferative disorder a therapeutically effective amount of a composition comprising an agent that interferes with LPA concentration and/or activity, preferably in a pharmaceutically or veterinarily acceptable carrier, as the intended application may require. LPA-associated hyperproliferative disorders include neoplasias, disorders associated with endothelial cell proliferation, and disorders associated with fibrogenesis. Most often, the neoplasia will be a cancer. Typical disorders associated with endothelial cell proliferation are angiogenesis-dependent disorders, for example, cancers caused by a solid tumors, hematological tumors, and age-related macular degeneration. Disorders associated with fibrogenesis include those than involve aberrant cardiac remodeling, such as cardiac failure.

There are many known hyperproliferative disorders, in which cells of various tissues and organs exhibit aberrant patterns of growth, proliferation, migration, signaling, senescence, and death. While a number of treatments have been developed to address some of these diseases, many still remain largely untreatable with existing technologies, while in other cases, while treatments are available, they are frequently less than optimal and are seldom curative.

1. Cancer

Cancer represents perhaps the most widely recognized class of hyperproliferative disorders. Cancers are a devastating class of diseases, and together, they have a mortality rate second only to cardiovascular disease. Many cancers are not fully understood on a molecular level. As a result, cancer is a major focus of research and development programs for both the United States government and pharmaceutical companies. The result has been an unprecedented R&D effort and the production of many valuable therapeutic agents to help in the fight against cancer.

Unfortunately the enormous amount of cancer research has not been enough to overcome the significant damage caused by cancer. There are still over one million new cases of cancer diagnosed annually and over five hundred thousand deaths in the United States alone. This is a dramatic demonstration that even though an enormous effort has been put forth to discover new therapeutics for cancer, effective therapeutic agents to combat the disease remain elusive.

Cancer is now primarily treated with one or a combination of three types of therapies, surgery, radiation, and chemotherapy. Surgery involves the bulk removal of diseased tissue. While surgery is sometimes effective in removing tumors located at certain sites, for example, in the breast, colon, and skin, it cannot be used in the treatment of tumors located in other areas, such as the backbone, nor in the treatment of disseminated neoplastic conditions such as leukemia. Radiation therapy involves the exposure of living tissue to ionizing radiation causing death or damage to the exposed cells. Side effects from radiation therapy may be acute and temporary, while others may be irreversible. Chemotherapy involves the disruption of cell replication or cell metabolism.

Further insult is that current therapeutic agents usually involve significant drawbacks for the patient in the form of toxicity and severe side effects. Therefore, many groups have recently begun to look for new approaches to fighting the war against cancer. These new so-called “innovative therapies” include gene therapy and therapeutic proteins such as monoclonal antibodies.

The first monoclonal antibody used in the clinic for the treatment of cancer was Rituxan (rituximab) which was launched in 1997, and has demonstrated the utility of monoclonal antibodies as therapeutic agents. Thus, not surprisingly, twenty monoclonal antibodies have since been approved for use in the clinic, including nine that are prescribed for cancer. The success of these products, as well as the reduced cost and time to develop monoclonal antibodies as compared with small molecules has made monoclonal antibody therapeutics the second largest category of drug candidates behind small molecules. Further, the exquisite specificity of antibodies as compared to small molecule therapeutics has proven to be a major advantage both in terms of efficacy and toxicity. For cancer alone there are currently more than 270 industry antibody R&D projects with more than 50 companies involved in developing new cancer antibody therapeutics. Consequently, monoclonal antibodies are poised to become a major player in the treatment of cancer and they are estimated to capture an increasing share of the cancer therapeutic market. Generally therapeutic mAbs are targeted to proteins; only recently has it been feasible to raise mAbs to bioactive lipids (for example, antibodies to S1P, see Applicants' US Application Serial No. 20070148168).

The identification of extracellular mediators that promote tumor growth and survival is a critical step in discovering therapeutic interventions that will reduce the morbidity and mortality of cancer. As described below, LPA is considered to be a pleiotropic, tumorigenic growth factor. LPA promotes tumor growth by stimulating cell proliferation, cell survival, and metastasis. LPA also promotes tumor angiogenesis by supporting the migration and survival of endothelial cells as they form new vessels within tumors. Taken together, LPA initiates a proliferative, pro-angiogenic, and anti-apoptotic sequence of events contributing to cancer progression. Thus, therapies that modulate, and, in particular, reduce LPA levels in vivo will be effective in the treatment of cancer.

Typically, the methods of the invention for treating or preventing a hyperproliferative disorder such as cancer involve administering to a subject suffering from a hyperproliferative disorder an effective amount of each of an agent (or a plurality of different agent species) according to the invention and a cytotoxic agent. Cytotoxic agents include chemotherapeutic drugs.

A related aspect concerns methods of reducing toxicity of a therapeutic regimen for treatment or prevention of a hyperproliferative disorder. Such methods comprise administering to a subject suffering from a hyperproliferative disorder an effective amount of an agent (or a plurality of different agent species) according to the invention before, during, or after administration of a therapeutic regimen intended to treat or prevent the hyperproliferative disorder. It is believed that by sensitizing cells, e.g., cancer cells, to chemotherapeutic drugs, efficacy can be achieved at lower doses and hence lower toxicity due to chemotherapeutic drugs.

Yet another aspect of the invention concerns methods of enhancing a survival probability of a subject treated for a hyperproliferative disorder by administering to a subject suffering from a hyperproliferative disorder an agent (or a plurality of different agent species) according to the invention before, during, or after administration of a therapeutic regimen intended to treat or prevent the hyperproliferative disorder to enhance the subject's survival probability.

2. Fibrosis, Wound Healing and Scar Formation

Fibroblasts, particularly myofibroblasts, are key cellular elements in scar formation in response to cellular injury and inflammation (Tomasek et al. (2002), Nat Rev Mol Cell Biol, vol 3: 349-63, and Virag and Murry (2003), Am J Pathol, vol 163: 2433-40). Collagen gene expression by myofibroblasts is a hallmark of remodeling and necessary for scar formation (Sun and Weber (2000), Cardiovasc Res, vol 46: 250-6, and Sun and Weber (1996), J Mol Cell Cardiol, vol 28: 851-8).

Fibrosis can be described as the formation or development of excess or aberrant fibrous connective tissue in an organ or tissue as part of a pathological reparative or reactive process, in contrast to normal wound healing or development. The most common forms of fibrosis are: liver, lung, kidney, skin, uterine and ovarian fibroses. Some conditions, such as scleroderma, sarcoidosis and others, are characterized by fibrosis in multiple organs and tissues.

Recently, the bioactive lysophospholipid lysophosphatidic acid (LPA) has been recognized for its role in tissue repair and wound healing. Watterson et al., Wound Repair Regen. (2007) 15:607-16. As a biological mediator, LPA has been recognized for its role in tissue repair and wound healing (Watterson, 2007). In particular, LPA is linked to pulmonary and renal inflammation and fibrosis. LPA is detectable in human bronchioalveolar lavage (BAL) fluids at baseline and its expression increases during allergic inflammation Georas, S. N. et al. (2007) Clin Exp Allergy. (2007) 37: 311-22. Furthermore, LPA promotes inflammation in airway epithelial cells. Barekzi, E. et al (2006) Prostaglandins Leukot Essent Fatty Acids. 74:357-63. Recently, pulmonary and renal fibrosis have been linked to increased LPA release and signaling though the LPA type 1 receptor (LPA₁). LPA levels were elevated in bronchialveolar lavage (BAL) samples from IPF patients and bleomycin-induced lung fibrosis in mice was dependent on activation of LPA₁. Tager et al., (2008) Proc Am Thorac Soc. 5: 363. (2008) Following unilateral ureteral obstruction in mice, tubulointerstitial fibrosis was reduced in LPA₁ knock-out mice and pro-fibrotic cytokine expression was attenuated in wild-type mice treated with an LPA₁ antagonist. J. P. Pradere et al., (2007) J. Am. Soc. Nephrol. 18:3110-3118. LPA has been shown to have direct fibrogenic effects in cardiac fibroblasts by stimulating collagen gene expression and proliferation. Chen, et al. (2006) FEBS Lett. 580:4737-45. Combined, these studies demonstrate a role for LPA in tissue repair and fibrosis, and identify bioactive lipids as a previously unrecognized class of targets in the treatment of fibrotic disorders.

a. Scleroderma

The compositions and methods of the invention will be useful in treating disorders and diseases characterized, at least in part, by aberrant neovascularization, angiogenesis, fibrogenesis, fibrosis, scarring, inflammation, and immune response. One such disease is scleroderma, which is also referred to as systemic sclerosis.

Scleroderma is an autoimmune disease that causes scarring or thickening of the skin, and sometimes involves other areas of the body, including the lungs, heart, and/or kidneys. Scleroderma is characterized by the formation of scar tissue (fibrosis) in the skin and organs of the body, which can lead to thickening and firmness of involved areas, with consequent reduction in function. Today, about 300,000 Americans have scleroderma, according to the Scleroderma Foundation. One-third or less of those affected have widespread disease, while the remaining two-thirds primarily have skin symptoms. When the disease affects the lungs and causing scarring, breathing can become restricted because the lungs can no longer expand as they should. To measure breathing capability, doctors use a device that assesses forced vital capacity (FVC). In people with an FVC of less than 50 percent of the expected reading, the 10-year mortality rate from scleroderma-related lung disease is about 42 percent. One reason the mortality rate is so high is that no effective treatment is currently available.

Without wishing to be bound by any particular theory, it is believed that inappropriate concentrations of lipids such as S1P and/or LPA, and/or their metabolites, cause or contribute to the development of scleroderma. As such, the compositions and methods of the invention can be used to treat scleroderma, particularly by decreasing the effective in vivo concentration of a particular target lipid, for example, LPA.

Evidence indicates that LPA is a pro-fibrotic growth factor that can contribute to fibroblast activation, proliferation, and the resulting increased fibroblast activity associated with maladaptive scarring and remodeling. Moreover, potential roles for LPA in skin fibroblast activity have been demonstrated. For example, it has been shown that LPA stimulates the migration of murine skin fibroblasts (Hama et al., J Biol Chem. 2004 Apr. 23; 279(17):17634-9).

b. Pulmonary Fibrosis

Pulmonary fibrosis, sometimes referred to as interstitial lung disease or ILD, affects more than 5 million people worldwide. Within the USA the prevalence of the disease seems to be under-estimated and vary from 3 to 6 cases for 100,000 inhabitants to 28 per 100,000. Within Europe; the numbers vary depending on the countries, and is reported around 1 to 24 cases per 100,000 without a clear gender effect. The disease is usually diagnosed between 40 and 70 years of age. The median survival is 3 to 5 years. Despite its prevalence, there are no therapies available to halt or reverse the progression of IPF and there are no FDA-approved courses of treatment. Thus, there is an unmet need for new therapeutic strategies to treat IPF as well as other diseases that involve pathological tissue fibrosis.

Interstitial lung disease, or ILD, includes more than 180 chronic lung disorders, which are chronic, nonmalignant and noninfectious. Interstitial lung diseases are named for the tissue between the air sacs of the lungs called the interstitium—the tissue affected by fibrosis (scarring). Interstitial lung diseases may also be called interstitial pulmonary fibrosis or pulmonary fibrosis. The symptoms and course of these diseases may vary from person to person, but the common link between the many forms of ILD is that they all begin with an inflammation, e.g.: bronchiolitis—inflammation that involves the bronchioles (small airways); alveolitis—inflammation that involves the alveoli (air sacs); vasculitis—inflammation that involves the small blood vessels (capillaries)

More than 80% of interstitial lung diseases are diagnosed as pneumoconiosis, drug-induced disease, or hypersensitivity pneumonitis. The other types are:

Occupational and environmental exposures: Many jobs, particularly those that involve working with asbestos, ground stone, or metal dust, can cause pulmonary fibrosis. The small particles are inhaled, damage the alveoli, and cause fibrosis. Some organic substances, such as moldy hay can also initiate pulmonary fibrosis; this is known as farmer's lung.

Asbestosis is usually caused when small needle-like particles of asbestos are inhaled into the lungs. This can cause lung scarring (pulmonary fibrosis) and in addition can lead to lung cancer. The key to asbestosis is prevention. In manufacturing asbestos products, both employer and employee must be aware of government standards and should take all precautions against inhaling the particles. The paramount danger in working with asbestos comes when old, friable (crumbly) asbestos-containing products are replaced or destroyed. In those circumstances, particles can be released into the air and breathed into the lungs. Today however, the asbestos fibres usually are “locked in” by binders such as cement, rubber or plastics, thus preventing the particles from floating free in the air. Cigarette smoking has an interactive relationship with asbestos—the asbestos worker who smokes has a much higher chance of developing lung cancer than does the non-smoker.

Silicosis is another disease producing pulmonary fibrosis in which the cause is known. It is a disease that results from breathing in free crystalline silica dust. All types of mining in which the ore is extracted from quartz rock can produce silicosis if precautions are not taken. This includes the mining of gold, lead, zinc, copper, iron, anthracite (hard) coal, and some bituminous (soft) coal. Workers in foundries, sandstone grinding, tunneling, sandblasting, concrete breaking, granite carving, and china manufacturing also encounter silica.

Large silica particles are stopped in the upper airways. But the tiniest specks of silica can be carried down to the alveoli where they lead to pulmonary fibrosis. Silicosis can be either mild or severe, in direct proportion to the percentage and concentration of silica in the air and the duration of exposure. Silicosis can be prevented by measures specifically designed for each industry and each job. Dust control is essential. Sometimes this is accomplished by the wetting down of mines, improved ventilation, or the wearing of masks.

Idiopathic pulmonary fibrosis: Although a number of separate diseases can initiate pulmonary fibrosis, many times the cause is unknown. When this is so, the condition is called “idiopathic (of unknown origin) pulmonary fibrosis”. In idiopathic pulmonary fibrosis, careful examination of the patient's environmental and occupational history gives no clues to the cause. Some physicians and scientists believe that the disease is an infectious or allergic condition, however bacteria and other microorganisms are not routinely found in the lungs of such patients. On the other hand, the condition does sometimes appear to follow a viral-like illness. Thus, although the cause of pulmonary fibrosis is known in many cases, the idiopathic variety still remains a mystery.

Sarcoidosis is disease characterized by the formation of granulomas (areas of inflammatory cells), which can attack any area of the body but most frequently affects the lungs.

Certain medicines may have the undesirable side effect of causing pulmonary fibrosis; for example, Nitrofurantoin (sometimes used for urinary tract infections); Amiodarone (sometimes prescribed for an irregular heart rate); Bleomycin, cyclophosphamide, and methotrexate (sometimes prescribed to fight cancer).

Radiation, such as given as treatment for breast cancer, may also cause pulmonary fibrosis. Other diseases characterized, at least in part, by pulmonary fibrosis include tuberculosis, rheumatoid arthritis, systemic lupus erythematosis, systemic sclerosis, grain handler's lung, mushroom worker's lung, bagassosis, detergent worker's lung, maple bark stripper's lung, malt worker's lung, paprika splitter's lung, bird breeder's lung and Hermansky Pudlak syndrome. Pulmonary fibrosis can also be genetically inherited.

Clinical Features:

Breathlessness is the hallmark of pulmonary fibrosis. Many lung diseases show breathlessness as the main symptom—a fact that can complicate and confuse diagnosis. Usually the breathlessness idiopathic pulmonary fibrosis first appears during exercise. The condition may progress to the point where any exertion is impossible. A dry cough is a common symptom. The fingertips may enlarge at the ends and take on a bulbous appearance. This is often referred to as “clubbing”.

Additional symptoms may include: shortness of breath, especially with exertion, fatigue and weakness, loss of appetite, loss of weight, dry cough that does not produce phlegm, discomfort in chest, labored breathing and hemorrhage in lungs.

Diagnosis

In addition to a complete medical history and physical examination, the following tests maybe required to refine and/or confirm the diagnosis of pulmonary fibrosis: pulmonary function tests—to determine characteristics and capabilities of the lungs; spirometry—to measure the amount of air that can be forced out; peak flow meter—to evaluate changes in breathing and response to medications; blood tests—to analyze the amount of carbon dioxide and oxygen in the blood; X-ray; computerized axial tomography (CAT) scan; bronchoscopy—to examine the lung using a long, narrow tube called a bronchoscope; bronchoalveolar lavage—to remove cells from lower respiratory tract to help identify inflammation and exclude certain causes; and lung biopsy—to remove tissue from the lung for examination in the pathology laboratory.

Treatment

If one of the known causes of pulmonary fibrosis exists, then treatment of that underlying disease or removal of the patient from the environment causing the disease can be effective. This may include treatment with: oral medications, including corticosteroids; influenza vaccine; pneumococcal pneumonia vaccine, oxygen therapy from portable tanks and/or lung transplantation.

Many times treatment is limited only to treating the inflammatory response that occurs in the lungs. This is done in the hope that stopping the inflammation will prevent the laying down of scar tissue or fibrosis in the lungs and thus stop the progression of the disease.

Corticosteroids are the drugs which are usually administered in an attempt to stop the inflammation. The advantage of this treatment has not been proven in every case, although it does appear that if the drugs are given early on in the course of the disease, there is a better chance of improvement. Corticosteroid medications can have various side effects and so patients taking these medications must be frequently reassessed by their physicians in order to judge the safety and benefit of this therapy.

Other drugs have been tried but convincing evidence of their efficacy is lacking. Although drug therapy of pulmonary fibrosis may not always be successful, there is much that can be done in the way of supportive therapy that will ease the breathlessness that accompanies this condition. Rehabilitation and education programs can help considerably in teaching patients how to breathe more efficiently and to perform their activities of daily living with less breathlessness. Sometimes supplemental oxygen therapy is required in order to treat breathlessness. Early treatment of chest infections is required. Smoking must be discontinued, as the effects of tobacco will aggravate the shortness of breath.

Outcome

Many times the disease is mild with few symptoms and does not progress significantly with the years. In other cases, when pulmonary fibrosis is due to some other underlying disease such as rheumatoid arthritis, progression of the lung condition may reflect progression of the underlying diseases. Very rarely pulmonary fibrosis has a sudden onset and rapidly progresses to death from respiratory failure over a period of weeks. However, the usual course of pulmonary fibrosis, particularly idiopathic pulmonary fibrosis, is one of slowly progressive scarring of the lungs. The duration and speed of this process is variable. Some patients respond to therapy. In other cases, patients do not respond to therapy and have a slow deterioration over months to years, eventually ending in death when lungs can no longer function adequately.

LPA and Pulmonary Fibrosis

Although the exact etiology is not known, IPF is believed to result from an aberrant wound healing response following pulmonary injury. Scotton, C. J. and Chambers, R. C. (2007) Chest, 132:1311-21. In particular, increased proliferation and migration of lung fibroblasts as well as the formation of scar tissue-producing myofibroblasts are key events in the pathogenesis of IPF. Myofibroblasts are smooth muscle-like fibroblasts that express alpha-smooth muscle actin (α-SMA) and contain a contractile apparatus composed of actin filaments and associated proteins that are organized into prominent stress fibers. In addition to their normal role in tissue homeostasis and repair, myofibroblasts are pathological mediators in numerous fibrotic disorders. Hinz, B. (2007) J Invest Dermatol. 127:526-37. Increased number and density of myofibroblasts has been demonstrated in the fibrotic foci of animal models of lung fibrosis. Myofibroblasts are formed following tissue injury whereby increased levels of growth factors, cytokines and mechanical stimuli promote transformation of resident tissue fibroblasts into contractile, scar tissue-producing myofibroblasts. In the lung and other tissues, persistent, elevated levels of biochemical mediators including TGFβ, CTGF, PDGF and various inflammatory cytokines, promotes myofibroblast formation and exaggerated scar tissue production which leads to tissue fibrosis (Scotton, 2007). Thus, current clinical strategies for treating IPF and other fibrotic disorders have targeted biochemical factors that promote myofibroblast formation and subsequent fibrous tissue production.

Recently, the bioactive lysophospholipid lysophosphatidic acid (LPA) has been recognized for its role in tissue repair and wound healing (Watterson, 2007). LPA is a bioactive lysophospholipid (<500 Dalton) with a single hydrocarbon backbone and a polar head group containing a phosphate group. LPA elicits numerous cellular effects through the interaction with specific G protein-coupled receptors (GPCR), designated EGD2/LPA₁, EDG4/LPA₂, EDG7/LPA₃, and LPA₄. Anliker B. and J. Chun, (2004) Seminars in Cell & Developmental Biology, 15: 457-465. As a biological mediator, LPA has been recognized for its role in tissue repair and wound healing (Watterson, 2007). In particular, LPA is linked to pulmonary and renal inflammation and fibrosis. LPA is detectable in human bronchioalveolar lavage (BAL) fluids at baseline and its expression increases during allergic inflammation (Georas, 2007). Furthermore, LPA promotes inflammation in airway epithelial cells (Barekzi, 2006). Recently, pulmonary and renal fibrosis have been linked to increased LPA release and signaling though the LPA type 1 receptor (LPA₁). LPA levels were elevated in bronchialveolar lavage (BAL) samples from IPF patients and bleomycin-induced lung fibrosis in mice was dependent on activation of LPA₁ (Tager, 2008). Following unilateral ureteral obstruction in mice, tubulointerstitial fibrosis was reduced in LPA₁ knock-out mice and pro-fibrotic cytokine expression was attenuated in wild-type mice treated with an LPA₁ antagonist (Pradere, 2007). Combined, these studies demonstrate a role for LPA in tissue repair and fibrosis, and identify bioactive lipids as a previously unrecognized class of targets in the treatment of IPF and other fibrotic disorders.

c. Hepatic (Liver) Fibrosis

The liver possesses a remarkable regenerative capacity, therefore the process of repair by regeneration proceeds to complete restitutio ad integrum (full restoration). If however the damage has affected the reticular framework, the repair will occur by scar formation (fibrosis) which may lead to rearrangement of the blood circulation and to cirrhosis.

The reaction to injury proceeds as is follows: Damage (necrosis), accompanied by cellular changes and tissue changes; inflammatory reaction; and repair (either by regeneration (restitutio ad integrum) or by scarring (fibrosis).

Chronic liver diseases lead to fibrosis which leads to disturbance of the architecture, portal hypertension and may produce such an irreversible rearrangement of the circulation as to cause cirrhosis. There is a fine line between fibrosis and cirrhosis. Fibrosis is not only the result of necrosis, collapse and scar formation but also the result of disturbances in the synthesis and degradation of matrix by injured mesenchymal cells that synthesize the various components of the matrix which in the liver are the following categories: collagens, glycoproteins and proteoglycans.

Evaluation of Liver Fibrosis

Evaluation of Liver Fibrosis can be histological, e.g., with Masson trichrome stain, silver reticulin stain, specific antibodies for collagen types, desmin and vimentin for lipocytes, or vimentin for myofibroblasts, or may be biochemical, e.g, by: determination of various enzymes in matrix or of serum laminin in benign fibrosis.

Classifications of Liver Fibrosis

There are 2 main types, congenital and acquired liver fibrosis. The former is a genetic disorder, which causes polycystic liver diseases. The latter has many different categories and is mainly caused by liver cell injuries. Pathologically, fibrosis can be classified as:

Portal area fibrosis: There is fibroblasts proliferation and fibers expansion from the portal areas to the lobule. Finally, these fibers connected to form bridging septa. This kind of fibrosis is mainly seen in viral hepatitis and malnutritional liver fibrosis.

Intra-lobular fibrosis: There is almost no fibroblast found in normal lobule. When large numbers of liver cells degenerate and undergo necrosis, the reticular fiber frame collapses and becomes thick collagen fibers. At the same time, intra lobule fibrotic tissue proliferates and surrounds the liver cells.

Central fibrosis: Proliferated fibrotic tissue mainly surrounds the center vein and causes the thickening of the wall of the center vein.

Peri-micro-bile-duct fibrosis: Type fibrosis mainly caused by long-term bile retention and mainly happens around the bile ducts. Microscopically, there are connective tissues surrounding the newly formed bile canaliulus and bile-plugs. The base-membrane of the bile canaliulus becomes fibrotic.

Immunologically, liver fibrosis can be classified as:

Passive fibrosis: There is extensive necrosis of the liver cells and secondary liver structure collapse and scar formation, which causes connective tissue proliferation.

Active fibrosis: Lymph cells and other inflammatory cells infiltration and recurrent and consistent inflammation promote the connective tissue to invade the lobule.

Causally, liver fibrosis can be classified as:

Viral hepatitis fibrosis: Usually caused by chronic hepatitis B, C, and D. Worldwide, there are three hundred fifty million of hepatitis B virus carriers, and one hundred seventy million of hepatitis C infected people. About 15% of HBV and 85% of HCV infected persons will develop chronic hepatitis and lead to fibrosis. In which, the liver shows peri-portal area inflammation and piecemeal necrosis and fibrosis. With such large population being affected, this is the most important category of the liver fibrosis.

Parasitic infection fibrosis: This kind of liver fibrosis is mainly happening in developing countries and is caused by schistosomiasis. There are two hundred and twenty million people in Asia, Africa, South and Center America suffering from this infection. The recurrent infection and the eggs of schistosome accumulated in the liver can cause liver fibrosis and cirrhosis.

Alcoholic fibrosis: It is mainly caused by the oxidized metabolite of alcohol, acetaldehyde. In western countries, the incidence of this disorder is positively related to the amount of alcohol consumption. The total cases of alcoholic fibrosis in the USA is about three times higher than the number of hepatitis C. Alcoholic fibrosis causes two morphological changes in the liver: fatty liver and cellular organelles deterioration. The fibrosis first appears around the center veins and at the same time, the liver parenchymal inflammation. Gradually the fibrosis expends to the whole liver.

Biliary fibrosis: There is primary and secondary biliary fibrosis. Primary biliary hepatic fibrosis (PBHF) is an autoimmune disorder in which chronic intra-liver bile retention caused the liver fibrosis. It is more often affect female around the age 40 to 60. In serum tests, elevated gamma globulin and positive for the anti-mitochondria antibody. Pathological studies found that the fibrosis mainly around the micro-bile ducts and periportal area fibrosis and inflammation. Secondary biliary fibrosis happens following the obstruction of the bile ducts, which causes peri-portal inflammation and progressive fibrosis.

Metabolic fibrosis: This category is not common and has fewer cases. Wilson's disease or liver lenticular degeneration and hemochromatosis are the main disorders that cause metabolic fibrosis. The former is a genetic disorder and causes cooper metabolism disorder and deposits in the liver. The latter is an iron metabolic disorder and causes hemoglobin deposits in the liver. Both of these metabolic disorders can cause liver fibrosis and cirrhosis.

Intoxication fibrosis: When long-term contact with liver-toxic substances, such as carbon-tetrachloride, organophosphorus, dimethyl nitrosamine, thioacetamide, or taking liver toxic medications, such as isoniazid, thio-oxidizing pyrimidine, wintermin, tetracycline, acetaminophen etc. can all cause various degrees of liver cell injuries, necrosis, bile retention, or allergic inflammation and cause liver fibrosis.

Malnutritional fibrosis: This type is mainly caused by insufficient or imbalanced nutritional intake. A long-term low protein or high fat diet can cause fatty liver and lead to fibrosis.

Cardiogenic fibrosis: Chronic congestive heart failure can cause long lasting liver vein stagnancy causing ischemic degeneration of the liver cells. In this type of liver fibrosis, the connective tissue hypertrophy starts at the center of the liver lobule and gradually expands to rest of the lobule.

Diagnosis and Staging of Liver Fibrosis

The gold standard for assessing the health of the liver is the liver biopsy. However since the procedure requires that a needle be inserted through the skin there is a potential for complications even though the incidence of complications is extremely low. The complications of a liver biopsy can include internal bleeding, and puncturing another organ such as the lungs, stomach, intestines, or any other organs that are close to the liver. In regards to accuracy of the biopsy the sample liver tissue size is important for correctly staging and grading a liver biopsy. Another problem is that the tissue taken from one part of the liver may not be 100% representative of the entire liver. Once the liver tissue sample is collected it is graded and staged by a specialist (pathologist), which could lead to possible human error in interpreting the results. In addition there is no standardized interpretation protocol so it is difficult to compare the results of different biopsies read by different pathologists. Price is also an issue since a typical liver biopsy can cost between $1,500 and $2,000.

Given these potential problems it is not surprising that there is a lot of research that is being conducted on the development of non-invasive tests. The tests that have been developed so far have had mixed results in accuracy when compared to the results of a liver biopsy. There have been few prospective clinical trials that have compared the results from various non-invasive markers to the results from a liver biopsy.

In order to objectively evaluate the stage of fibrosis, liver biopsy, especially a series of biopsies, is the main method used today. From the biopsy, it is possible to diagnose the liver inflammation grade and also the stage of the fibrosis. The most commonly used scoring system is Kanel scoring system, which stages the fibrosis from 0 to 5. (At the same time the biopsy diagnosis also give a ranking of inflammation grade, which is from 0 to 4) Stage 0: normal; Stage 1: portal expansion with fibrosis (<⅓ tracts with wisps of bridging.); Stage 2: bridging fibrosis; Stage 3: marked bridging fibrosis or early cirrhosis (with thin septa fibrosis); Stage 4: definite cirrhosis with <50% of biopsy fibrosis; Stage 5: definite cirrhosis with >50% of biopsy fibrosis.

Blood tests to diagnose liver fibrosis: Because biopsy is an invasive procedure, many patients are wary of the procedure. Blood tests are being studied as a method to evaluate the fibrosis progression. The most commonly used serum chemical analysis method is by measuring the amount of HA (hyaluronic acid), LN (Laminin), CIV (collagen IV), PCIII (procollagen type III) in the serum. They can be used as a reference index of fibrosis activities. From the blood tests, the ratio of AST/ALT is found and when it is greater than 1, it often shows that the degree of fibrosis is relatively advanced. Combined with whether is there an enlarged spleen and depletion of platelets count and albumin level, we can also estimate the stage of the fibrosis. In advanced fibrosis, the spleen is usually enlarged with platelets counts lower than 100 and albumin lower than 3.5. With blood test results, the evaluation of the severity of fibrosis is only useful to access the stage 0, 1 and 3, 4, and 5. It is not able to distinguish the stages between 2 and 3.

Medical imagery diagnosis B-ultrasonic, CT, and MRI can also be used to evaluate the liver fibrosis. The B-ultrasonic image is often used to check the size of the spleen, measure the diameter of the main stern of the portal vein, the diameters of right and left portal vein branches, the diameter of vein at the portal of the spleen, and the blood flow speed of the portal vein. GI endoscopies can be used to see whether varices exists in the stomach and esophagus. These can be used as a reference for the hepatologist to evaluate the stage of fibrosis.

In general, the term fibrosis refers to the abnormal formation of fibrous (scar) tissue. For hepatitis patients, fibrosis means that the liver has been under assault by the hepatitis for some time. Early stages of fibrosis are identified by discrete, localized areas of scarring in one portal (zone) of the liver. Later stages of fibrosis are identified by “bridging” fibrosis, which is scar tissue that crosses across zones of the liver. The rate at which people progress from inflammation to fibrosis, and eventually to cirrhosis seems to vary tremendously, but in most people the progression is very slow. There is a growing body of evidence that people who respond to interferon therapy for HCV infection may experience a decrease in the amount of tissue scarring. This speaks to the liver's ability to regenerate itself. If fibrosis advances far enough, it is described as Cirrhosis. Liver biopsy is conducted to assess the degree of inflammation (grade) and degree of scarring (stage). Diagnosis: One of the major clinical problems facing the hepatology and gastroenterology community is how best to evaluate and manage the increasing numbers of patients identified with hepatitis C virus (HCV). In the last decade, advances in serologic and virologic testing for HCV and improvements in therapy have led more patients to be identified and to seek treatment. However, little progress has been made in improving either our ability to determine the degree of hepatic injury, particularly fibrosis, or to predict the risk of disease progression for the individual patient.

The clinician relies on the biopsy results for both prognostic and therapeutic decision making, which can have a major impact on the patient's life. A single-pass liver biopsy is able to correctly diagnose the stage of fibrosis or presence of cirrhosis in 80% of patients. Factors that improve the diagnostic accuracy of liver biopsy include the presence of a uniform disease throughout the liver such as HCV, multiple passes, type of needle used, and an unfragmented biopsy core of 2 cm or greater in length. Even with experienced physicians performing the liver biopsy and expert pathologists interpreting the biopsy, this gold standard has up to a 20% error rate in staging disease.

d. Renal (Kidney) Fibrosis

LPA is linked to renal inflammation and fibrosis. Recently, renal fibrosis has been linked to increased LPA release and signaling though the LPA type 1 receptor (LPA₁). Following unilateral ureteral obstruction in mice, tubulointerstitial fibrosis was reduced in LPA₁ knock-out mice and pro-fibrotic cytokine expression was attenuated in wild-type mice treated with an LPA₁ antagonist (Pradere, 2007).

e. Other Fibroses

Uterine fibroses are non-malignant tumors known as uterine leiomyomata (commonly called fibroids). They can be isolated or grow in clusters, with sizes varying from the size of an apple seed to the size of a grapefruit or larger. Diagnosis of uterine fibroids is generally achieved by ultrasound, X-rays, CAT scan, laparoscopy and/or hysteroscopy. Treatment of uterine fibroids can be either medical (drug treatment, e.g., non-steroid anti-inflammatory drugs or gonadotropin release hormone agonists) or surgical (e.g., myomectomy, hysterectomy, endometrial ablation or myolysis, with recent development of less invasive methods such as uterine fibroid embolization and thermal ultrasound ablation.

Fibrosis of the skin can be described as a thickening or hardening of the skin, and occurs in scleroderma and other fibrotic skin diseases. When severe, fibrosis can limit movement and normal function. A keloid is an excessive scar that forms in response to trauma, sometimes minor trauma such as ear piercing or acne. Unlike normal scar formation, keloids have disproportionate proliferation of fibroblasts resulting in masses of collagenous tissue. The scar therefore protrudes above the surface of the surrounding skin and infiltrates skin which was not originally traumatized. Roles for LPA in skin fibroblast activity have been demonstrated. For example, it has been shown that LPA stimulates the migration of murine skin fibroblasts (Hama et al., J Biol Chem. 2004 Apr. 23; 279(17):17634-9). Thus it is believed that anti-LPA agents such as antibodies are useful for treatment of aberrant skin fibrosis such as keloids or skin fibrosis.

f. Cardiac Fibrosis

LPA has also been shown to have direct fibrogenic effects in cardiac fibroblasts by stimulating collagen gene expression and fibroblast proliferation. Chen, et al. (2006) FEBS Lett. 580:4737-45. Thus anti-LPA agents such as antibodies are expected to have anti-fibrotic effects in cardiac cells as well, and thus to be effective in treatment of cardiac fibrosis.

Agents that reduce the effective concentration of LPA, such as Lpath's anti-LPA mAb, are believed to be useful in methods for treating diseases and conditions characterized by aberrant fibrosis.

3. Cardiovascular and Cerebrovascular Disorders

Because LPA is involved in fibrogenesis and wound healing of liver tissue (Davaille et al., J. Biol. Chem. 275:34268-34633, 2000; Ikeda et al., Am J. Physiol. Gastrointest. Liver Physiol 279:G304-G310, 2000), healing of wounded vasculatures (Lee et al., Am. J. Physiol. Cell Physiol. 278:C612-C618, 2000), and other disease states, or events associated with such diseases, such as cancer, angiogenesis and inflammation (Pyne et al., Biochem. J. 349:385-402, 2000), the compositions and methods of the disclosure may be applied to treat not only these diseases but cardiac diseases as well, particularly those associated with tissue remodeling. LPA have some direct fibrogenic effects by stimulating collagen gene expression and proliferation of cardiac fibroblasts. Chen, et al. (2006) FEBS Lett. 580:4737-45.

b. Obesity and Diabetes

Autotaxin, a phospholipase D responsible for LPA synthesis, has been found to be secreted by adipocytes and its expression is up-regulated in adipocytes from obese-diabetic db/db mice as well as in massively obese women subjects and human patients with type 2 diabetes, independently of obesity (Ferry et al. (2003) JBC 278:18162-18169; Boucher et al. (2005) Diabetologia 48:569-577, cited in Pradere et al. (2007) BBA 1771:93-102. LPA itself has been shown to influence proliferation and differentiation of preadipocytes. Pradere et al., 2007. Together this suggests a role for anti-LPA agents in treatment of obesity and diabetes.

c. Pain

Pain is the most common reason for doctor visits in the US and is present as part of a broad spectrum of diseases, disorders and conditions. Pain may be acute or chronic and may be classified according to location in the body and/or by etiology, although in many cases the etiology of pain is not understood or may be due to several possible causes, which may overlap. Pain may also be described qualitatively, as allodynia (abnormal sensory perception of pain) or hyperalgesia (exaggerated pain sensations), for example.

Neuropathic pain is a complex, often chronic form of pain associated with damage or dysfunction of the nervous system Simply stated, neuropathic pain is a chronic pain state caused by pathological changes in the nervous system. Myers, et al (2006) Drug Disc. Today 11: 8-20. Causes of acute and/or chronic neuropathic pain include, but are not limited to, injury, trauma, or damage to the central or peripheral nervous system (e.g., spinal cord injury, disc herniation, multiple sclerosis or other degenerative or neurodegenerative disease), inflammation, drug exposure (for example, cytotoxics such as Taxol, cisplatin, and other chemotherapeutic agents), diabetes, viral disease (such as, for example, HIV and herpes zoster), metabolic disease, severe ischemic insults, nutrient deficiency, toxin exposure, and cancer. Cancer neuropathic pain may result directly from tumor impingement on nerves, or indirectly such as from radiation, surgery, or drug treatment. Neuropathic pain is mediated through neuroinflammatory mechanisms controlled by inflammatory responses to the initial insult and affecting nervous system tissue. Myers, et al (2006), Drug Disc. Today 11: 8-20. Many inflammatory mediators, such as TNFα, have been found to be pivotal in neuropathic pain. Leung L, Cahill C M. (2010) J Neuroinflamm., 7:27. Neuropathic pain is unresponsive to most common painkillers.

Inflammatory mediators are involved in the genesis, persistence, and severity of pain. IL-6 is a potent pain-generating inflammatory mediator. IL-6 is produced in the rat spinal cord following peripheral nerve injury, with levels of IL-6 levels correlating directly with the intensity of allodynia. Arruda, et al. (2000), Brain Res. 879:216-25. IL-6 levels increase during stress or inflammation, and rheumatoid arthritis is associated with increased levels of IL-6 in synovial fluid. Matsumoto, et al (2006), Rheumatol. Int. 26:1096-1100; Desgeorges, et al. (1997), J. Rheumatol. 24:1510-1516. Neuropathic pain is prevented in IL-6 knockout mice. Xu, et al (1997), Cytokine 9:1028-1033.

IL-8 is a pain-generating inflammatory mediator. Drug treatment of post-herpetic neuralgia showed a decrease of 50% in IL-8 concentrations, and this decrease correlated with pain relief. Kotani, et al. (2000), New Engl. J. Med. 343:1514-1519.

TNF-α induces axonal damage, macrophage recruitment and ectopic activity in peripheral nerve fibers and plays a role in the generation of hyperalgesia. TNFα is upregulated at the site of peripheral nerve lesions and in patients with neuropathic pain. Thalidomide, a selective blocker of TNF production, reduces hyperalgesia in an animal model of neuropathic pain (chronic constriction injury). George, et al. (2000), Pain 88:267-275.

A significant role of LPA in the development of neuropathic pain was established using various pharmacological and genetic approaches. LPA is responsible for long-lasting mechanical allodynia and thermal hyperalgesia as well as demyelination and upregulation of pain-related proteins through the LPA₁ receptor. In addition, intrathecal injections of LPA induce behavioral, morphological, and biochemical changes such as prolonged sensitivity to pain stimuli accompanied by demyelination of dorsal roots, similar to those observed after nerve ligation. Fujita, R., Kiguchi, N. & Ueda, H. (2007), Neurochem Int 50, 351-5. Wild-type animals with nerve injury develop behavioral allodynia and hyperalgesia paralleled by demyelination in the dorsal root and increased expression of both the protein kinase C isoform within the spinal cord dorsal horn and the 21 calcium channel subunit in dorsal root ganglia. It has been demonstrated that mice lacking the LPA1 receptor gene (lpa1−/− mice) lose nerve injury-induced neuropathic pain behaviors and phenomena. Inoue, et al. (2004), Nat Med 10, 712-8. Heterozygous mutant mice for the autotaxin gene (atx+/−) showed approximately 50% recovery of nerve injury-induced neuropathic pain. The hyperalgesia was completely abolished in both lpa1−/− and atx+/− mice. Furthermore, inhibitors of Rho and Rho kinase signaling pathways also prevented neuropathic pain. Mueller, B. K., Mack, H. & Teusch, N. (2005), Nat Rev Drug Discov 4, 387-98. Therefore, targeting LPA biosynthesis and/or LPA1 receptor represents a novel, patentable approach to mitigating nerve-injury-induced neuropathic pain.

At the cellular level, LPA is a potent inducer of morphological changes in neuronal and glial cells. Kingsbury, et al. (2003), Nat Neurosci 6, 1292-9; Jalink, et al. (1993), Cell Growth Differ 4, 247-55; Tigyi, G. & Miledi, R. (1992), J Biol Chem 267, 21360-7 (1992); Fukushima, et al. (2000), Dev Biol 228, 6-18; Yuan, X. B. et al. (2003) Nat Cell Biol 5, 38-45; Fukushima, et al. (2007), Neurochem Int 50, 302-7.

In primary astrocytes, as well as in glioma-derived cell lines, LPA causes reversal of process outgrowth (‘stellation’), a process directed by active RhoA and accompanied by reassembly and activation of focal adhesion proteins. Ramakers, G. J. & Moolenaar, W. H. (1998), Exp Cell Res, 245: 252-62. A role for LPA in myelination is also suggested by the finding that LPA promotes cell-cell adhesion and survival in Schwann cells. Weiner, et al. (2001), J Neurosci. 21:7069-78; Ramer, et al (2004), J Neurosci. 24:10796-805.

3. Antibody Generation and Characterization

The instant invention relates to use of anti-LPA antibodies in the treatment of pain. The generation and characterization of murine and humanized monoclonal antibodies to LPA has been described in several patent applications, including U.S. Patent Application Publication No. 20100034814, which is commonly owned with the instant application and is incorporated herein in its entirety.

4. Pharmaceutical Formulations, Dosing and Routes of Administration

One way to control the amount of undesirable LPA in a patient is by providing a composition that comprises one or more anti-LPA antibodies to bind one or more LPAs, thereby acting as therapeutic “sponges” that reduce the level of free undesirable LPA. When a compound is stated to be “free,” the compound is not in any way restricted from reaching the site or sites where it exerts its undesirable effects. Typically, a free compound is present in blood and tissue, which either is or contains the site(s) of action of the free compound, or from which a compound can freely migrate to its site(s) of action. A free compound may also be available to be acted upon by any enzyme that converts the compound into an undesirable compound.

Anti-LPA antibodies may be formulated in a pharmaceutical composition that is useful for a variety of purposes, including the treatment of diseases, disorders or physical trauma. Pharmaceutical compositions suitable for antibodies to bioactive lipids are disclosed in US application publication US20100098700, which is commonly assigned with the instant application and is incorporated herein in its entirety.

Pharmaceutical compositions comprising one or more anti-LPA antibodies of the invention may be incorporated into kits and medical devices for such treatment. Medical devices may be used to administer the pharmaceutical compositions of the invention to a patient in need thereof, and according to one embodiment of the invention, kits are provided that include such devices. Such devices and kits may be designed for routine administration, including self-administration, of the pharmaceutical compositions of the invention. Such devices and kits may also be designed for emergency use, for example, in ambulances or emergency rooms, or during surgery, or in activities where injury is possible but where full medical attention may not be immediately forthcoming (for example, hiking and camping, or combat situations).

Therapeutic formulations of the antibody are prepared for storage by mixing the antibody having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™, or polyethylene glycol (PEG).

The formulation herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished for instance by filtration through sterile filtration membranes.

Sustained-release preparations may be prepared. Suitable examples of sustained-release preparations include semipermeable matrices of solid hydrophobic polymers containing the antibody, which matrices are in the form of shaped articles, e.g., films, or microcapsule. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and γ-ethyl-L-glutamate, non-degradable ethylene-vinyl acetate, degradable lactic acid-glycolic acid copolymers such as the Lupron Depot™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

For therapeutic applications, the anti-LPA agents, e.g., antibodies, of the invention are administered to a mammal, preferably a human, in a pharmaceutically acceptable dosage form such as those discussed above, including those that may be administered to a human parenterally (including intravenous, intraarterial, subcutaneous, intraperitoneal or intramuscular injection or infusion) or by intracranial, intrathecal, intra-cerebrospinal, subcutaneous, intra-articular, intrasynovial, oral, topical, intratracheal or inhalation routes.

For the prevention or treatment of disease, the appropriate dosage of antibody will depend on the type of disease to be treated, as defined above, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments.

Depending on the type and severity of the disease, about 1 μg/kg to about 50 mg/kg (e.g., 0.1-20 mg/kg) of antibody is an initial candidate dosage for administration to the patient, whether, for example, by one or more separate administrations, or by continuous infusion. A typical daily or weekly dosage might range from about 1 μg/kg to about 20 mg/kg or more, depending on the factors mentioned above. For repeated administrations over several days or longer, depending on the condition, the treatment is repeated until a desired suppression of disease symptoms occurs. However, other dosage regimens may be useful. The progress of this therapy is easily monitored by conventional techniques and assays, including, for example, radiographic imaging. Detection methods using the antibody to determine LPA levels in bodily fluids or tissues may be used in order to optimize patient exposure to the therapeutic antibody.

According to another embodiment of the invention, the composition comprising an agent, e.g, a mAb, that interferes with LPA activity is administered as a monotherapy, while in other preferred embodiments, the composition comprising the agent that interferes with LPA activity is administered as part of a combination therapy. In some cases the effectiveness of the antibody in preventing or treating disease may be improved by administering the antibody serially or in combination with another agent that is effective for those purposes, such as a chemotherapeutic drug for treatment of cancer or a conventional analgesic.

Such other agents may be present in the composition being administered or may be administered separately. Also, the antibody is suitably administered serially or in combination with the other agent or modality.

5. Kits and Articles of Manufacture

In another aspect of the invention, an article of manufacture containing materials useful for the treatment of pain is provided. The article of manufacture or kit comprises a container and a label. Suitable containers include, for example, bottles, vials, syringes, and test tubes. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). The active agent in the composition is the anti-LPA antibody. The label on, or associated with, the container indicates that the composition is used for treating the condition of choice. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, syringes, and package inserts with instructions for use.

The invention will be better understood by reference to the following Examples, which are intended to merely illustrate the best mode now known for practicing the invention. The scope of the invention is not to be considered limited thereto.

### EXAMPLES

The invention will be further described by reference to the following detailed examples. These Examples are in no way to be considered to limit the scope of the invention in any manner

### Example 1: Monoclonal Antibodies to LPA

Murine monoclonal antibodies to LPA were made as described in U.S. Patent Application Publication No. 20100034814, which is commonly owned with the instant application and is incorporated herein in its entirety and for all purposes. Six hybridoma clones were selected for characterization based on their superior biochemical and biological properties. Mouse hybridoma cell lines 504B3-6C2, 504B7.1, 504B58/3F8, 504A63.1 and 504B3A6 (corresponding to clones referred to herein as B3, B7, B58, A63, and B3A6, respectively) were received on May 8, 2007 by the American Type Culture Collection (ATCC Patent Depository, 10801 University Blvd., Manassas, Va. 20110) for patent deposit purposes on behalf of LPath Inc. and were granted deposit numbers PTA-8417, PTA-8420, PTA-8418, PTA-8419 and PTA-8416, respectively. All anti-LPA antibodies and portions thereof referred to herein were derived from these cell lines.

Direct Binding Kinetics

The binding of 6 anti-LPA mAbs (B3, B7, B58, A63, B3A6, D22) to 12:0 and 18:1 LPA (0.1 uM) was measured by ELISA. EC₅₀ values were calculated from titration curves using 6 increasing concentrations of purified mAbs (0 to 0.4 ug/ml). EC₅₀ represents the effective antibody concentration with 50% of the maximum binding. Max denotes the maximal binding (expressed as OD450). Results are shown in Table 1, below.

TABLE 1
Direct Binding Kinetics of Anti-LPA mAbs

| | | B3 | B7 | B58 | D22 | A63 | B3A6 |
|---|---|---|---|---|---|---|---|
| 12:0 LPA | EC₅₀ (nM) | 1.420 | 0.413 | 0.554 | 1.307 | 0.280 | 0.344 |
| | Max (OD450) | 1.809 | 1.395 | 1.352 | 0.449 | 1.269 | 1.316 |
| 18:1 LPA | EC₅₀ (nM) | 1.067 | 0.274 | 0.245 | 0.176 | 0.298 | 0.469 |
| | Max (OD450) | 1.264 | 0.973 | 0.847 | 0.353 | 1.302 | 1.027 |

The kinetics parameters kₐ (association rate constant), k_{d} (disassociation rate constant) and K_{D} (association equilibrium constant) were determined for the 6 lead candidates using the BIAcore 3000 Biosensor machine. In this study, LPA was immobilized on the sensor surface and the anti-LPA mAbs were flowed in solution across the surface. As shown, all six mAbs bound LPA with similar K_{D} values ranging from 0.34 to 3.8 pM and similar kinetic parameters.

The Anti-LPA Murine mAbs Exhibit High Affinity to LPA

LPA was immobilized to the sensor chip at densities ranging 150 resonance units. Dilutions of each mAb were passed over the immobilized LPA and kinetic constants were obtained by nonlinear regression of association/dissociation phases. Errors are given as the standard deviation using at least three determinations in duplicate runs. Results are shown in Table 2, below. Apparent affinities were determined by K_{D}=kₐ/k_{d}.

TABLE 2
Affinity of anti-LPA mAb for LPA

| mAbs | kₐ (M⁻¹ s⁻¹) | k_{d} (s⁻¹) | K_{D} (pM) |
|---|---|---|---|
| A63 | 4.4 ± 1.0 × 10⁵ | 1 × 10⁻⁶ | 2.3 ± 0.5 |
| B3 | 7.0 ± 1.5 × 10⁵ | 1 × 10⁻⁶ | 1.4 ± 0.3 |
| B7 | 6.2 ± 0.1 × 10⁵ | 1 × 10⁻⁶ | 1.6 ± 0.1 |
| D22 | 3.0 ± 0.9 × 10⁴ | 1 × 10⁻⁶ | 33 ± 10 |
| B3A6 | 1.2 ± 0.9 × 10⁶ | 1.9 ± 0.4 × 10⁻⁵ | 16 ± 1.2 |

kₐ = Association rate constant in M⁻¹s⁻¹
k_{d} = Dissociation rate constant in s⁻¹

Specificity Profile of Six Anti-LPA mAbs.

Many isoforms of LPA have been identified to be biologically active and it is preferable that the mAb recognize all of them to some extent to be of therapeutic relevance. The specificity of the anti-LPA mAbs was evaluated utilizing a competition assay in which the competitor lipid was added to the antibody-immobilized lipid mixture.

Competition ELISA assays were performed with the anti-LPA mAbs to assess their specificity. 18:1 LPA was captured on ELISA plates. Each competitor lipid (up to 10 uM) was serially diluted in BSA (1 mg/ml)-PBS and then incubated with the mAbs (3 nM). Mixtures were then transferred to LPA coated wells and the amount of bound antibody was measured with a secondary antibody. Data are normalized to maximum signal (A450) and are expressed as percent inhibition. Assays were performed in triplicate. IC₅₀: Half maximum inhibition concentration; MI: Maximum inhibition (% of binding in the absence of inhibitor); ---: not estimated because of weak inhibition. A high inhibition result indicates recognition of the competitor lipid by the antibody. As shown in Table 3, below, all the anti-LPA mAbs recognized the different LPA isoforms.

TABLE 3
Specificity profile of anti-LPA mAbs.

| | 14:0 LPA | | 16:0 LPA | | 18:1 LPA | | 18:2 LPA | | 20:4 LPA | |
|---|---|---|---|---|---|---|---|---|---|---|
| | IC₅₀ | MI | IC₅₀ | MI | IC₅₀ | MI | IC₅₀ | MI | IC₅₀ | MI |
| | uM | % | uM | % | uM | % | uM | % | uM | % |
| B3 | 0.02 | 72.3 | 0.05 | 70.3 | 0.287 | 83 | 0.064 | 72.5 | 0.02 | 67.1 |
| B7 | 0.105 | 61.3 | 0.483 | 62.9 | >2.0 | 100 | 1.487 | 100 | 0.161 | 67 |
| B58 | 0.26 | 63.9 | 5.698 | >100 | 1.5 | 79.3 | 1.240 | 92.6 | 0.304 | 79.8 |
| B104 | 0.32 | 23.1 | 1.557 | 26.5 | 28.648 | >100 | 1.591 | 36 | 0.32 | 20.1 |
| D22 | 0.164 | 34.9 | 0.543 | 31 | 1.489 | 47.7 | 0.331 | 31.4 | 0.164 | 29.5 |
| A63 | 1.147 | 31.9 | 5.994 | 45.7 | — | — | — | — | 0.119 | 14.5 |
| B3A6 | 0.108 | 59.9 | 1.151 | 81.1 | 1.897 | 87.6 | — | — | 0.131 | 44.9 |

Interestingly, the anti-LPA mAbs were able to discriminate between 12:0 (lauroyl), 14:0 (myristoyl), 16:0 (palmitoyl), 18:1 (oleoyl), 18:2 (linoleoyl) and 20:4 (arachidonoyl) LPAs. A desirable EC₅₀ rank order for ultimate drug development is 18:2>18:1>20:4 for unsaturated lipids and 14:0>16:0>18:0 for the saturated lipids, along with high specificity. The specificity of the anti-LPA mAbs was assessed for their binding to LPA related biolipids such as distearoyl-phosphatidic acid, lysophosphatidylcholine, S1P, ceramide and ceramide-1-phosphate. None of the antibodies demonstrated cross-reactivity to distearoyl PA and LPC, the immediate metabolic precursor of LPA.

### Example 2: Cloning of the Murine Anti-LPA Antibodies-Overview

Chimeric antibodies to LPA were generated using the variable domains (Fv) containing the active LPA binding regions of one of three murine antibodies from hybridomas with the Fc region of a human IgG1 immunoglobulin. As those in the art will appreciate, “humanized” antibodies can be generated by grafting the complementarity determining regions (CDRs, e.g. CDR1-4) of the murine anti-LPA mAbs with human antibody framework regions (e.g., Fr1, Fr4, etc.) such as the framework regions of an IgG1.

The overall strategy for cloning of the murine mAb against LPA consisted of cloning the murine variable domains of both the light chain (VL) and the heavy chain (VH) from each antibody. The consensus sequences of the genes show that the constant region fragment is consistent with a gamma isotype and that the light chain is consistent with a kappa isotype. The murine variable domains were cloned together with the constant domain of the human antibody light chain (CL) and with the constant domain of the human heavy chain (CH1, CH2, and CH3), resulting in a chimeric antibody construct. This process and the resulting chimeric antibodies are described in further detail in U.S. Patent Application Publication No. 20100034814, which is commonly owned with the instant application and is incorporated herein in its entirety. The mouse VH and VL domains were cloned and sequenced using standard methods, as described in U.S. Patent Application Publication No. 20100034814.

Tables 4-8, below, show amino acid sequences for the complementarity-determining regions (CDRs) of the variable (V_{H} and V_{L}) domains for five mouse anti-LPA monoclonal antibody clones. For each CDRH1 amino acid sequence, the CDR defined according to Kabat is the 10-amino acid sequence shown. The five-amino acid portion of the Kabat sequence that is shown in bold is the canonical CDRH1 sequence. Corresponding nucleic acid sequences are found in U.S. Patent Application Publication No. 20100034814.

TABLE 4
Mouse LPA CDR amino acid sequences of the
mouse V_{H} and V_{L} domains for clone B3 of mouse
anti-LPA monoclonal antibody

| CLONE | | CDR | SEQ ID NO: |
|---|---|---|---|
| | V_{H} CDR | | |
| B3 | GDAFTNYLIE* | CDRH1 | 1 |
| B3 | LIYPDSGYINYNENFKG | CDRH2 | 2 |
| B3 | RFAYYGSGYYFDY | CDRH3 | 3 |
| | V_{L} CDR | | |
| B3 | RSSQSLLKTNGNTYLH | CDRL1 | 4 |
| B3 | KVSNRFS | CDRL2 | 5 |
| B3 | SQSTHFPFT | CDRL3 | 6 |

*The CDRH1 sequence defined according to Chothia/AbM is the 10-amino acid sequence shown. The five-amino acid portion of this sequence shown in bold (NYLIE; SEQ ID NO: 7) is the CDRH1 sequence defined according to Kabat.

TABLE 5
Mouse LPA CDR amino acid sequences of the
mouse V_{H} and V_{L} domains for clone B7 of
mouse anti-LPA monoclonal antibody

| CLONE | | CDR | SEQ ID NO: |
|---|---|---|---|
| | V_{H} CDR | | |
| B7 | GYGFINYLIE* | CDRH1 | 8 |
| B7 | LINPGSDYTNYNENFKG | CDRH2 | 9 |
| B7 | RFGYYGSGNYFDY | CDRH3 | 10 |
| | V_{L} CDR | | |
| B7 | TSGQSLVHINGNTYLH | CDRL1 | 11 |
| B7 | KVSNLFS | CDRL2 | 12 |
| B7 | SQSTHFPFT | CDRL3 | 6 |

*The CDRH1 sequence defined according to Chothia/AbM is the 10-amino acid sequence shown. The five-amino acid portion of this sequence shown in bold (NYLIE; SEQ ID NO: 7) is the CDRH1 sequence defined according to Kabat.

TABLE 6
Mouse LPA CDR amino acid sequences of the
mouse V_{H} and V_{L} domains for clone B58 of
mouse anti-LPA monoclonal antibody

| CLONE | | CDR | SEQ ID NO: |
|---|---|---|---|
| | V_{H} CDR | | |
| B58 | GDAFTNYLIE* | CDRH1 | 1 |
| B58 | LIIPGTGYTNYNENFKG | CDRH2 | 13 |
| B58 | RFGYYGSSNYFDY | CDRH3 | 14 |
| | V_{L} CDR | | |
| B58 | RSSQSLVHSNGNTYLH | CDRL1 | 15 |
| B58 | KVSNRFS | CDRL2 | 5 |
| B58 | SQSTHFPFT | CDRL3 | 6 |

*The CDRH1 sequence defined according to Chothia/AbM is the 10-amino acid sequence shown. The five-amino acid portion of this sequence shown in bold (NYLIE; SEQ ID NO: 7) is the CDRH1 sequence defined according to Kabat.

TABLE 7
Mouse LPA CDR amino acid sequences of the
mouse V_{H} and V_{L} domains for clone 3A6 of
mouse anti-LPA monoclonal antibody

| CLONE | | CDR | SEQ ID NO: |
|---|---|---|---|
| | V_{H} CDR | | |
| 3A6 | GDAFTNYLIE* | CDRH1 | 1 |
| 3A6 | LIIPGTGYTNYNENFKG | CDRH2 | 13 |
| 3A6 | RFGYYGSGYYFDY | CDRH3 | 16 |
| | V_{L} CDR | | |
| 3A6 | RSSQSLVHSNGNTYLH | CDRL1 | 15 |
| 3A6 | KVSNRFS | CDRL2 | 5 |
| 3A6 | SQSTHFPFT | CDRL3 | 6 |

*The CDRH1 sequence defined according to Chothia/AbM is the 10-amino acid sequence shown. The five-amino acid portion of this sequence shown in bold (NYLIE; SEQ ID NO: 7) is the CDRH1 sequence defined according to Kabat.

TABLE 8
Mouse LPA CDR amino acid sequences of the
mouse V_{H} and V_{L} domains for clone A63 of
mouse anti-LPA monoclonal antibody

| CLONE | | CDR | SEQ ID NO: |
|---|---|---|---|
| | V_{H} CDR | | |
| A63 | GFSITSGYYWT* | CDRH1 | 17 |
| A63 | YIGYDGSNDSNPSLKN | CDRH2 | 18 |
| A63 | AMLRRGFDY | CDRH3 | 19 |
| | V_{L} CDR | | |
| A63 | SASSSLSYMH | CDRL1 | 20 |
| A63 | DTSKLAS | CDRL2 | 21 |
| A63 | HRRSSYT | CDRL3 | 22 |

*The CDRH1 sequence defined according to Chothia/AbM is the 11-amino acid sequence shown. The six-amino acid portion of this sequence shown in bold (SGYYWT; SEQ ID NO: 23) is the CDRH1 sequence defined according to Kabat.

Tables 9-13 below show the amino acid sequences of the murine anti-LPA antibody variable domains.

TABLE 9
Clone B3 variable domain amino acid sequences
without leader sequence and cut sites

| | SEQ |
|---|---|
| | ID |
| Sequence | NO: |
| B3 Heavy Chain | |
| QVKLQQSGPELVRPGTSVKVSCTASGDAFTNYLIEWVKQRPGQG | 24 |
| LEWIGLIYPDSGYINYNENFKGKATLTADRSSSTAYMQLSSLTSE | |
| DSAVYFCARRFAYYGSGYYFDYWGQGTTLTVSS | |
| B3 Light Chain | |
| DVVMTQTPLSLPVSLGDQASISCRSSQSLLKTNGNTYLHWYLQKP | 25 |
| GQSPKLLIFKVSNRFSGVPDRFSGSGSGTDFTLKISRVEAEDLGVY | |
| FCSQSTHFPFTFGTGTKLEIK | |

TABLE 10
Clone B7 variable domain amino acid sequences
without leader sequence and cut sites

| | SEQ |
|---|---|
| | ID |
| Sequence | NO: |
| B7 Heavy Chain | |
| QVQLQQSGAELVRPGTSVKVSCKASGYGFINYLIEWIKQRPGQGL | 26 |
| EWIGLINPGSDYTNYNENFKGKATLTADKSSSTAYMHLSSLTSED | |
| SAVYFCARRFGYYGSGNYFDYWGQGTTLTVSS | |
| B7 Light Chain | |
| DVVMTQTPLSLPVSLGDQASISCTSGQSLVHINGNTYLHWYLQKP | 27 |
| GQSPKLLIYKVSNLFSGVPDRFSGSGSGTDFTLKISRVEAEDLGVY | |
| FCSQSTHFPFTFGTGTKLEIK | |

TABLE 11
Clone B58 variable domain amino acid sequences
without leader sequence and cut sites

| | SEQ |
|---|---|
| | ID |
| Sequence | NO: |
| B58 Heavy Chain | |
| QVQLQQSGAELVRPGTSVKVSCKASGDAFTNYLIEWVKQRPGQG | 28 |
| LEWIGLIIPGTGYTNYNENFKGKATLTADKSSSTAYMQLSSLTSE | |
| DSAVYFCARRFGYYGSSNYFDYWGQGTTLTVSS | |
| B58 Light Chain | |
| DVVMTQTPLSLPVSLGDQASISCRSSQSLVHSNGNTYLHWYLQK | 29 |
| PGQSPKLLIYKVSNRFSGVPDRFSGSGPGTDFTLKISRVEAEDLG | |
| IYFCSQSTHFPFTFGTGTKLEIK | |

TABLE 12
Clone 3A6 variable domain amino acid sequences
without leader sequence and cut sites

| | SEQ |
|---|---|
| | ID |
| Sequence | NO: |
| 3A6 Heavy Chain | |
| QVQLQQSGAELVRPGTSVKLSCKASGDAFTNYLIEWVKQRPGQG | 30 |
| LEWIGLIIPGTGYTNYNENFKGKATLTADKSSSTAYMQLSSLTSE | |
| DSAVYFCARRFGYYGSGYYFDYWGQGTTLTVSS | |
| 3A6 Light Chain | |
| DVVMTQTPLSLPVSLGDQASISCRSSQSLVHSNGNTYLHWYLQK | 31 |
| PGQSPKLLIYKVSNRFSGVPDRFSGSGPGTDFTLKISRVEAEDLG | |
| VYFCSQSTHFPFTFGTGTKLEIK | |

TABLE 13
Clone A63 variable domain amino acid sequences
without leader sequence and cut sites

| | SEQ |
|---|---|
| | ID |
| Sequence | NO: |
| A63 Heavy Chain | |
| DIQLQESGPGLVKPSQSLSLTCSVTGFSITSGYYWTWIRQFPGNK | 32 |
| LEWVAYIGYDGSNDSNPSLKNRISITRDTSKNQFFLKLNSVTTED | |
| TATYYCARAMLRRGFDYWGQGTTLTVSS | |
| A63 Light Chain | |
| QIVLTQSPAIMSASPGEKVTMTCSASSSLSYMHWYQQKPGTSPKR | 33 |
| WIYDTSKLASGVPARFSGSGSGTSYSLTISSMEAEDAATYYCHRR | |
| SSYTFGGGTKLEIK | |

### Example 3: Murine Antibody B7

Murine antibody clone B7 has high affinity for the signaling lipid LPA (K_{D} of 1-50 pM as demonstrated by surface plasmon resonance in the BiaCore assay, and in a direct binding ELISA assay); in addition, B7 demonstrates high specificity for LPA, having shown no binding affinity for over 100 different bioactive lipids and proteins, including over 20 bioactive lipids, some of which are structurally similar to LPA. The murine antibody is a full-length IgG1k isotype antibody composed of two identical light chains and two identical heavy chains with a total molecular weight of 155.5 kDa. The biophysical properties are summarized in Table 14, below.

TABLE 14
General Properties of Murine antibody B7

| | |
|---|---|
| Identity | B7 (also referred to as LT3000 or Lpathomab) |
| Antibody isotype | Murine IgG1k |
| Specificity | Lysophosphatidic acid (LPA) |
| Molecular weight | 155.5 kDa |
| OD of 1 mg/mL | 1.35 (solution at 280 nm) |
| K_{D} | 1-50 pM |
| Apparent Tm | 67° C. at pH 7.4 |
| Appearance | Clear if dissolved in 1x PBS buffer (6.6 mM |
| | phosphate, 154 mM sodium chloride, pH 7.4) |
| Solubility | >40 mg/mL in 6.6 mM phosphate, 154 mM sodium |
| | chloride, pH 7.4 |

B7 has also shown biological activity in preliminary cell based assays such as cytokine release, migration and invasion; these are summarized in Table 15, below, along with data showing specificity of B7 for LPA isoforms and other bioactive lipids, and in vitro biological effects.

TABLE 15
LT3000 (B7 antibody)

| | 14:0 | 16:0 | 18:1 | 18:2 | 20:4 |
|---|---|---|---|---|---|
| A. Competitor Lipid | LPA | LPA | LPA | LPA | LPA |
| IC₅₀ (μM) | 0.105 | 0.483 | >2.0 | 1.487 | 0.161 |
| MI (%) | 61.3 | 62.9 | 100 | 100 | 67 |

| B. Competitor Lipid | LPC | S1P | C1P | Cer | DSPA |
|---|---|---|---|---|---|
| MI (%) | 0 | 2.7 | 1.0 | 1 | 0 |

| | LPA | |
|---|---|---|
| C. Cell based assay | isoform | |
| | | % Inhibition |
| | | (over LPA taken as 100) |
| Migration | 18:1 | 35* |
| Invasion | 14:0 | 95* |
| IL-8 Release | 18:1 | 20 |
| IL-6 Release | 18:1 | 23* |
| | | % Induction |
| | | (over LPA + TAXOL taken as 100) |
| Apoptosis | 18:1 | 79 |

A. Competition ELISA assay was performed with B7 and 5 LPA isoforms. 18:1 LPA was captured on ELISA plates. Each competitor lipid (up to 10 μM) was serially diluted in BSA/PBS and incubated with 3 nM B7. Mixtures were then transferred to LPA coated wells and the amount of bound antibody was measured.
B. Competition ELISA was performed to assess specificity of B7. Data were normalized to maximum signal (A₄₅₀) and were expressed as percent inhibition (n = 3). IC₅₀: half maximum inhibition concentration; MI %: maximum inhibition (% of binding in the absence of inhibitor).
C. Migration assay: B7 (150 μg/mL) reduced SKOV3 cell migration triggered by 1 μM LPA (n = 3); Invasion assay: B7 (15 mg/mL) blocked SKOV3 cell invasion triggered by 2 μM LPA (n = 2); Cytokine release of human IL-8 and IL-6: B7 (300-600 μg/mL, respectively) reduced 1 μM LPA-induced release of pro-angiogenic and metastatic IL-8 and IL-6 in SKOV3 conditioned media (n = 3). Apoptosis: SKOV3 cells were treated with 1 μM Taxol; 1 μM LPA blocked Taxol induced caspase-3 activation. The addition to B7 (150 μg/mL) blocked LPA-induced protection from apoptosis (n = 1). Data Analysis: Student-t test, * denotes p < 0.05.

The potent and specific binding of B7/LT3000 to LPA results in reduced availability of extracellular LPA (decrease in effective concentration of LPA) with potentially therapeutic effects.

A second murine anti-LPA antibody, B3, was also subjected to binding analysis as shown in Table 16, below.

TABLE 16
Biochemical characteristics of B3 antibody

| | High density | Low density |
|---|---|---|
| A. BIACORE | surface | surface |
| Lipid Chip | 12:0 LPA | 18:0 LPA |
| K_{D} (pM), site 1 (site2) | 61 (32) | 1.6 (0.3) |

| B. Competition Lipid Cocktail | |
|---|---|
| (C₁₆:C₁₈:C_{18:1}:C_{18:2}:C_{20:4}, | |
| ratio 3:2:5:11:2) | (μM) |
| IC₅₀ | 0.263 |

| C. Neutralization Assay | |
|---|---|
| B3 antibody (nmol) | LPA (nmol) |
| 0 | 0.16 |
| 0.5 | 0.0428 |
| 1 | 0.0148 |
| 2 | under limit of detection |

A. Biacore analysis for B3 antibody. 12:0 and 18:0 isoforms of LPA were immobilized onto GLC sensor chips; solutions of B3 were passed over the chips and sensograms were obtained for both 12:0 and 18:0 LPA chips. Resulted sensograms showed complex binding kinetics of the antibody due to monovalent and bivalent antibody binding capacities. K_{D} values were calculated approximately for both LPA 12 and LPA 18.
B. Competition ELISA assay was performed with B3 and a cocktail of LPA isoforms (C₁₆:C₁₈:C_{18:1}:C_{18:2}:C_{20:4} in ratio 3:2:5:11:2). Competitor/Cocktail lipid (up to 10 μM) was serially diluted in BSA/PBS and incubated with 0.5 μg/mL B3. Mixtures were then transferred to a LPA coated well plate and the amount of bound antibody was measured. Data were normalized to maximum signal (A₄₅₀) and were expressed as IC₅₀ (half maximum inhibition concentration).
C. Neutralization assay: Increasing concentrations of B3 were conjugated to a gel. Mouse plasma was then activated to increase endogenous levels of LPA. Activated plasma samples were then incubated with the increasing concentrations of the antibody-gel complex. LPA leftover which did not complex to the antibody was then determined by ELISA. LPA was sponged up by B3 in an antibody concentration dependent way.

### Example 4: Humanization of Lpathomab (B7, LT3000)

The variable domains of the murine anti-LPA monoclonal antibody B7 were humanized by grafting the murine CDRs into human framework regions (FR), as fully described in U.S. Patent Application Publication No. 20100034814 and U.S. patent application Ser. No. 12/761,584 and foreign equivalent PCT/US10/31339, which are commonly assigned with the instant application, and the contents of which are incorporated herein in their entirety, with the goal of producing an antibody that retains high affinity, specificity and binding capacity for LPA.

Engineering of the Humanized Variants

The murine anti-LPA antibody was humanized by grafting of the Kabat CDRs from LT3000 VH and VL into acceptor human frameworks. Seven humanized variants were transiently expressed in HEK 293 cells in serum-free conditions, purified and then characterized in a panel of assays. Plasmids containing sequences of each light chain and heavy chain were transfected into mammalian cells for production. After 5 days of culture, the mAb titer was determined using quantitative ELISA. All combinations of the heavy and light chains yielded between 2-12 ug of antibody per ml of cell culture.

A three-dimensional (3D) model containing the humanized VL and VH sequences was constructed to identify FR residues juxtaposed to residues that form the CDRs. These FR residues potentially influence the CDR loop structure and the ability of the antibody to retain high affinity and specificity for the antigen. Based on this analysis, 6 residues in AJ002773 and 3 residues in DQ187679 were identified, deemed significantly different from LT3000, and considered for mutation back to the murine sequence. Framework selection and backmutation identification was conducted by DataMabs, LLP, Radlett, Hertfordshire, UK. A list of the humanized variants is summarized in Table 17, below. The I2V mutation, which is present within the light chain of every variant studied, supports the presentation of residues in the CDRL3. Other light chain back mutations include Q45K, which is solvent exposed, and the conservative Y87F mutation, located on the side of the variable domain opposite the CDRs. Based on their position, the heavy chain back mutations appear more likely to influence the stability and LPA-binding properties of the mAb. I24A and V28G support residues that form the CDRH1 and the cluster of back mutations (I37V, M48I, V67A and I69L) form an elaborate network of hydrophobic interactions that likely effect the stability of the folded variable domain and the position of the CDRH2. The role of these back mutations on LPA binding, thermostability and cytokine released were investigated to identify the lead candidate for development of a fully humanized, anti-LPA monoclonal antibody.

TABLE 17
Vector designation and expression level of the chimeric and the humanized variants in
HEK293 cells.

| | Light Chain | | Heavy Chain | | Culture V | Expression |
|---|---|---|---|---|---|---|
| mAb | pATH | Back mutations | pATH | Back mutations | ml | (ug/ml) |
| LT3010 | 510 | none | 610 | None | 30 | 8.44 |
| LT3011 | 502 | I2V, Q45K, Y87F | 603 | S24A, I28G, M48I | 60 | 2.88 |
| LT3012 | 502 | I2V, Q45K, Y87F | 604 | I28G, M48I, | 30 | 11.2 |
| | | | | V67A, I69L | | |
| LT3013 | 506 | I2V | 603 | S24A, 128G, M481 | 60 | 5.33 |
| LT3014 | 506 | I2V | 604 | I28G, M48I, | 60 | 5.83 |
| | | | | V67A, I69L | | |
| LT3015 | 502 | I2V, Q45K, Y87F | 602 | S24A, I28G, V37I, | 60 | 5.99 |
| | | | | M48I, V67A, I69L | | |
| LT3016 | 506 | I2V | 602 | S24A, I28G, V37I, | 60 | 3.74 |
| | | | | M481, V67A, I69L | | |

Expression of the Humanized Variants

The humanized variants shown in the table above were transiently expressed in HEK 293 cells in serum-free conditions, purified and then characterized in a panel of assays. Plasmids containing sequences of each light chain (pATH500 series) and heavy chain (pATH600 series) were transfected into mammalian cells for production. After 5 days of culture, the mAb titer was determined using quantitative ELISA. All combinations of the heavy and light chains yielded between 2-12 ug of antibody per ml of cell culture. SDS-PAGE under reducing conditions revealed two bands at 25 kDa and 50 kDa with high purity (>98%), consistent with the expected masses of the light and heavy chains. A single band was observed under non-reducing conditions with the expected mass of ˜150 KDa.

Characterization of the Humanized Variants

The biophysical properties of the humanized variants were characterized for their binding affinity, binding capacity, yield, potency and stability. All the humanized anti-LPA mAb variants exhibited binding affinity in the low picomolar range similar to the chimeric anti-LPA antibody (also known as LT3010) and the murine antibody (LT3000). All of the humanized variants exhibited a T_{M} similar to or higher than that of LT3000, and most had a Tm of approximately 71° C. With regard to specificity, the humanized variants demonstrated similar specificity profiles to that of LT3000. For example, LT3000 demonstrated no cross-reactivity to lysophosphatidyl choline (LPC), phosphatidic acid (PA), various isoforms of lysophosphatidic acid (14:0 and 18:1 LPA, cyclic phosphatidic acid (cPA), and phosphatidylcholine (PC).

Activity of the Humanized Variants

Five humanized variants (LT3011, LT3013, LT3014, LT3015 and LT3016) were further assessed in in vitro cell assays. LPA is known to play an important role in eliciting the release of interleukin-8 (IL-8) from cancer cells. LT3000 reduced IL-8 release from ovarian cancer cells in a concentration-dependent manner. The humanized variants exhibited a similar reduction of IL-8 release compared to LT3000.

Some humanized variants were also tested for their effect on microvessel density (MVD) in a Matrigel tube formation assay for neovascularization. Both were shown to decrease MVD formation.

TABLE 18
Quantitation of microblood vessel density using CD31 immunostain with H&E
counterstaining in matrigel plugs.

| | | | | Humanized | Humanized | Humanized |
|---|---|---|---|---|---|---|
| | | | | variant #1 | variant #1 | variant #2 |
| | | LT3000 murine | LT3000 murine | (LT3015) | (LT3015) | (LT3016) |
| | Control | (8 mg/kg) | (2 mg/kg) | (8 mg/kg) | (2 mg/kg) | (2 mg/kg) |
| Average | 64.2 | 41.5 | 34 | 34.4 | 49 | 50.8 |
| S.E. | 8.0 | 14.2 | 13.7 | 4.2 | 31.5 | 18.8 |
| N= | 5 | 4 | 5 | 5 | 5 | 6 |

| | | | | | |
|---|---|---|---|---|---|
| Percent Inhibition | 35.4 | 47.0 | 46.4 | 23.7 | 20.8 |

Humanized anti-LPA antibody LT3015 (also referred to as “Lpathomab” was chosen for further characterization.

### Example 5: Preliminary Animal Pharmacokinetics of Lpathomab

Preliminary PK studies were conducted with Lpathomab. For IV dosed groups, mice were injected with a single 30 mg/kg dose and sacrificed at time points up to 15 days. Antibody was also given via i.p. administration and animals were sacrificed during the first 24 hrs to compare levels of mAb in the blood over this period of time for different routes of delivery. Pharmacokinetic parameters were assessed by WinNonlin. Three mice were sacrificed at each time point and plasma samples were collected and analyzed for mAb levels by ELISA. The half-life of Lpathomab in mice was determined to be 102 hrs (4.25 days) by i.v. administration. Moreover, the antibody is fully distributed to the blood within 6-12 hrs when given i.p., suggesting that the i.p. administration is suitable.

TABLE 19
Pharmacokinetic profile of Lpathomab in mice
Pharmacokinetic Parameters

| | Treatment | | | | | |
|---|---|---|---|---|---|---|
| Group | (mg/kg) | Route | | Estimate | SD | CV % |
| 1 | 30 | IV | AUC | 88.35 | 60.23 | 68.18 |
| | | | K10-HL | 102.7 | 77.48 | 75.91 |
| | | | Cmax | 0.6 | 0.13 | 21.71 |
| | | | Cl | 0.34 | 0.23 | 68.24 |
| | | | AUMC | 13009.8 | 18549.2 | 142.58 |
| | | | MRT | 147.25 | 111.78 | 75.91 |
| | | | Vss | 50 | 10.86 | 21.73 |

Software used to calculate the parameters: WinNonlin v1.1
AUG Area under the curve
K10-HL Elimination half-life
Cmax Dose related peak value
Cl Clearance
AUMC Area under the first moment curve
MRT Mean residence time
Vss Apparent volume of distribution, steady state

### Example 6: ANTI-LPA mAB Inhibits LPA-Induced IL-6 Release

IL-6 is a potent pain-generating inflammatory mediator. IL-6 is produced in the rat spinal cord following peripheral nerve injury, with levels of IL-6 levels correlating directly with the intensity of allodynia. Arruda, et al. (2000), Brain Res. 879:216-25. IL-6 levels increase during stress or inflammation, and rheumatoid arthritis is associated with increased levels of IL-6 in synovial fluid. Matsumoto, et al (2006), Rheumatol. Int. 26:1096-1100; Desgeorges, et al. (1997), J. Rheumatol. 24:1510-1516. Neuropathic pain is prevented in IL-6 knockout mice. Xu, et al (1997) Cytokine 9:1028-1033. In primary astrocytes, treatment with LPA (1 uM) causes IL-6 release. An experiment was conducted to evaluate the effect of anti-LPA antibody on IL-6 release in primary astrocytes.

Rat primary astrocytes were purchased from Cambrex (Charles City, Iowa) and cultured following vendor instructions. For IL-6 release assay, cells were seeded in a 96-well plate at the density of 1×104 cells per well and serum starved in media without serum for 24 hrs. After serum-starvation, primary astrocytes were treated with 1 mM LPA (solubilized in 1 mg/mL fatty acid-free BSA in PBS) previously incubated in the presence or absence of murine anti-LPA monoclonal antibody B3 (150 or 300 mg/mL antibody (1:1 or 1:2 molar ratio mAb:LPA; 1 hr at 37° C. in 5% CO2 humidified incubator). After 24 hr, conditioned media were collected and tested by human IL-6 ELISA (Rat IL-6 Quantikine Kit, R&D systems, Minneapolis Minn.) following vendor instructions. IL-6 values (pg/ml) were calculated using GraphPad software (La Jolla Calif.).

Cell conditioned media from human primary astrocytes were tested for IL-6 levels after 24 hrs of incubation with 1 mM LPA in presence or absence of molar ratio concentrations of B3 antibody (1:1 or 1:2 mAb:LPA). Treatment with LPA plus antibody to LPA (murine antibody B3) at a ratio of 2:1 not only blocked the IL-6 release but lowered IL-6 levels to approximately half the control level. LPA plus antibody at a ratio of 1:1 caused nearly as great a reduction in IL-6 levels. Thus anti-LPA antibody blocks the release of IL-6 that occurs in response to astrocyte treatment with LPA.

### Example 7: Antibody to LPA Reduces Allodynia in Diabetes-Induced Neuropathic Pain Model

Studies were performed in the rat model of streptozotocin-induced type 1 (insulin deficient) diabetes using tactile allodynia and hyperalgesia during the formalin test as behavioral indices of diabetes-induced neuropathic pain. All experimental procedures have been published. Calcutt N A, Freshwater J D, O'Brien J S (2000), Anesthesiology 93:1271-1278; Jolivalt C G, Ramos K M, Herbetsson K, Esch F S, Calcutt N A (2006), Pain 121:14-21.

Female Sprague-Dawley rats (Harlan Industries, San Diego Calif.) weighing 225-250 grams each were maintained at room temperature, between 65 to 82° F. with relative humidity between 30 to 70%. The room was illuminated with fluorescent lighting on a daily 12 hour light/dark cycle. All animals were maintained 2/cage with free access to dry food and municipal water.

Insulin deficient diabetes was induced following an overnight fast by a single IP injection of streptozotocin (55 mg/kg) dissolved in 0.9% sterile saline. Hyperglycemia was confirmed 4 days later and also prior to behavioral testing in a sample of blood obtained by tail prick using a strip operated reflectance meter. All animals were observed daily and weighed regularly during the study period.

Tactile Response Threshold:

Rats were transferred to a testing cage with a wire mesh bottom and allowed to acclimate. Von Frey filaments (Stoelting, Wood Dale Ill.) were used to determine the 50% mechanical threshold for foot withdrawal. A series of filaments, starting with one possessing a buckling weight of 2.0 g, were applied in sequence to the plantar surface of the right hindpaw with a pressure that causes the filament to buckle. Lifting of the paw was recorded as a positive response and the next lightest filament chosen for the next measurement. Absence of a response after 5 seconds prompted use of the next filament of increasing weight. This paradigm was continued until four measurements were made after an initial change in the behavior or until five consecutive negative (given the score of 15 g) or four positive (score of 0.25 g) scores occurred. The resulting sequence of positive and negative scores was used to interpolate the 50% response threshold. Only rats with a 50% tactile response threshold below 6 g were considered allodynic and brought forward for drug testing.

### Formalin Test:

Rats were restrained manually and formalin (50 μl of 0.2% or 0.5% solution) injected sub-dermally into the hindpaw dorsum. Rats were then placed in an observation chamber and flinching behaviors counted in 1-minute blocks every 5 minutes for 1 hour.

### Tissue Collection:

Blood was removed from restrained rats by tail prick to confirm hyperglycemia in diabetic rats using a strip-operated reflectance meter. Blood (0.3-0.5 ml per sample) can also be drawn into heparin-coated tubes on ice, centrifuged (1500 g, 2° C., 10 minutes), and plasma stored at −70° C. for subsequent assay. CSF (20-50 μl) was collected and stored at −70° C. Portions of the peripheral neuraxis and spinal cord were removed into fixative or stored at −70° C. at autopsy for subsequent assay.

### Experimental Design

Four groups of diabetic rats and 1 group of control rats were established and tested for allodynia after 1 week of hyperglycemia. Rats were implanted with an IT catheter and treatment was by both IV (tail vein) and IT injection. Rats received twice weekly treatment by each route (Mon/Thu for IT, Tues/Fri for IV) during weeks 2 and 3. Tactile allodynia was tested at the start of week 4 (3-4 days after the last treatment) and then, if tactile allodyna was present in treated rats, at 1, 3 and 6 hr after IT (Mon) and IV (Tues) treatments. Otherwise untreated diabetic rats received a single treatment with gabapentin with subsequent measurement of tactile allodynia at 1, 3 and 6 hr post-drug, to serve as a positive treatment control. At the conclusion of the study, all rats received a final injection of anti-LPA antibody (IV and/or IT route to be determined based upon tactile test data) or gabapentin at a chosen time before paw formalin injection (0.2%), with evoked flinching followed for up to 1 hour. Animals were euthanized at the end of formalin testing and tissue collected for storage as described above.

### Pilot Study (Preliminary Results)

Rats were treated for 2 weeks after 4 weeks of diabetes and the pain withdrawal threshold was determined. Nondiabetic mice were used as controls. Each treated animal received 10 mg/kg of intravenously administered B3 antibody twice a week. In addition, the low dose animal(s) received intrathecal administration of 2 ug total B3 antibody twice a week and the high dose animal(s) received intrathecal administration of 10 ug total B3 antibody twice a week. The results are shown in Table 20, below.

TABLE 20
Reduction of tactile allodynia in diabetic mice by anti-LPA antibody

| Contrl + | | | Diabetic + | | | Diabetic + | | | Diabetic + | | |
|---|---|---|---|---|---|---|---|---|---|---|---|
| Veh | C + R + V | C + L + V | Veh | D + R + V | D + L + V | Low dose | D + R + LD | D + L + LD | High dose | D + R + HD | D + L + HD |
| 1.0 | 15.00 | 11.70 | 5.0 | 6.66 | 3.33 | 8.0 | 15.00 | 11.70 | 10.1 | 5.37 | 3.67 |
| 1.1 | 15.00 | 15.00 | 5.1 | 2.20 | 3.58 | 9.0 | 4.25 | 8.61 | 11.0 | 6.42 | 6.58 |
| 1.2 | 11.70 | 15.00 | 6.0 | 4.98 | 2.81 | 9.1 | 11.70 | 15.00 | 12.1 | 15.00 | 15.00 |
| 2.0 | 9.86 | 8.61 | 6.1 | 11.70 | 2.37 | 10.0 | 11.70 | 8.61 | 7.0 | 6.42 | 6.66 |
| 2.1 | 15.00 | 15.00 | 7.1 | 4.47 | 6.42 | 11.1 | 15.00 | 15.00 | | | |
| | | | 12.0 | 1.99 | 2.37 | | | | | | |
| Median | 15.00 | 15.00 | median | 4.72 | 3.07 | median | 11.70 | 11.70 | median | 6.42 | 6.62 |
| mean | 13.31 | 13.06 | mean | 5.33 | 3.48 | mean | 11.53 | 11.78 | mean | 8.30 | 7.98 |
| SEM | 0.76 | 0.91 | SEM | 1.13 | 0.48 | SEM | 1.46 | 1.07 | SEM | 1.59 | 1.73 |

As can be seen from this preliminary study, the anti-LPA antibody B3 increased the paw withdrawal threshold in diabetic rats, indicating a reduction in allodynia in rats with diabetes-induced neuropathic pain.

### Example 8: Anti-LPA Antibody in Sciatic Nerve Injury Model of Neuropathic Pain

Lysophosphatidic acid (LPA) is an endogenous bioactive agent that mediates multiple cellular responses including proliferation, differentiation, angiogenesis, motility, and protection from apoptosis in a variety of cell types. LPA initiates neuropathic pain and underlying machineries through LPA1 receptor signaling in mice with partial sciatic nerve injury (Ueda at al., Nature Med, 10(7):712-8 2004). In fact, LPA1-null mice lose various nerve injury-induced neuropathic pain and its underlying mechanisms such as demyelination, down-regulation of myelin proteins and up-regulation of Caᵥ α2δ-1 and spinal PKCγ. The sciatic nerve injury-induced demyelination was observed in sciatic nerve (SCN) and dorsal root (DR), but not spinal nerve (SN), and the demyelination was abolished in DR, but not SCN in LPA1 receptor knock-out mice. When spinal slices were stimulated by substance P plus NMDA, but not by either one, there was a marked time-dependent increase in the levels of LPA, which was converted from newly produced lysophosphatidyl choline (LPC) through an action of autotaxin (Inoue, M. et al. (2008) J Neurochem, 107(6):1556-65). Thus, it is evident that intense stimulation of sensory fibers leads to LPA production, which in turn leads to a demyelination of dorsal root fibers. In addition to the sciatic nerve injury-induced model of peripheral neuropathic pain, spinal cord injury-induced central neuropathic pain and central stress-induced chronic pain models have been developed.

Anti-LPA antibody was assessed in a sciatic nerve injury-induced model of peripheral neuropathic pain (Seltzer, et al. (1990), Pain, 43(2), 205-218), and could also be assessed in models of spinal cord injury-induced central neuropathic pain and central stress-induced chronic pain.

### In Vivo Studies

Six-week-old male and female C57BL/6J mice weighing 18-22 g were used. These mice were individually kept in a room maintained at 24±2° C., humidity 60±5%, and ad libitum feeding of a standard laboratory diet and tap water before use.

### 1) Partial Sciatic Nerve Injury (PSNI) Model

Mice were deeply anesthetized with 50 mg/kg pentobarbital. The common sciatic nerve of the right (or left) hindlimb was exposed at the level of the high thigh through a small incision, and the dorsal one half of the nerve thickness was tightly ligated with a silk suture.

### 2) Spinal Cord Injury (SCI) Model

Under pentobarbital (50 mg/kg) anesthesia, the dorsal surface of the dura mater is exposed after laminectomy of mice at the ninth thoracic spinal vertebrae. Spinal cord injury is produced at spinal segment of T9 using a commercially available SCI device (40 kdyn using Infinite Horizon impactor, Precision Systems & Instrumentation, Fairfax Station VA).

### 3) Intermittent Cold Stress (ICS) Model

Two mice per group are kept in a cold room at 4±2° C. at 4:30 p.m. on day 1, feeding and agar instead of water. Mice are placed on a stainless steel mesh and covered with plexiglass cage. At 10:00 a.m. the next morning, mice are transferred to the normal temperature room at 24±2° C. After they are placed at the normal temperature for 30 min, mice are put in the cold room again for 30 min These processes are repeated until 4:30 p.m. Mice are then put in the cold room overnight. After the same treatments on the next day, mice are finally taken out from the cold room at 10:00 a.m.
Anti-LPA antibody (B3) was supplied at a minimum concentration of 0.2 μg/μL diluted in artificial cerebrospinal fluid (aCSF) comprising 125 mM NaCl, 3.8 mM KCl, 2.0 mM CaCl2, 01.0 mM MgCl2, 1.2 mM KH2PO4, 26 mM NaHCO3 and 10 mM D-glucose (pH 7.4).

### 1) PSNI Model

### Anti-LPA Antibody Injection:

The intrathecal (i.c.v. or i.t.) injections of anti-LPA antibody were performed free hand between spinal L5 and L6 segments. The i.c.v. or i.t. injections were given in a volume of 5 μl (1 μg).

### Nociception Test:

The paw pressure test was carried out using a digital von Frey apparatus test (Anesthesiometer, IITC Inc., Woodland Hills, USA). In this experiment, the threshold (in grams) of given pressure to cause the paw withdrawal behavior of mouse was evaluated. The thermal paw withdrawal test [Hargreaves, et al. (1988), Pain 32:77-88] was carried out using a thermal stimulus (IITC Inc., Woodland Hills, Calif., USA). These behavioral experiments were conducted in mice at 1, 3, 7 and 14 days postligation.

### Immunohistochemistry for Protein Kinase Cγ and Cαα2δ1:

After nociception test (day14), mice are deeply anesthetized with i.p. pentobarbital and perfused transcardially with K+ free PBS followed by 4% paraformaldehyde (PFA). The dorsal root ganglion (DRG) and spinal cord between L4-L5 segments is removed and post-fixed in 4% PFA. For immunostaining of γ isoform of protein kinase C (PKCγ) and Caα2δ1, the sections are then reacted with a rabbit polyclonal antibody. The sections are then incubated with a FITC-conjugated anti-rabbit IgG.

### Toluidin Blue Staining and Transmission Electron Microscopy for Demyelination

DR fibers are fixed with 2.5% glutaraldehyde. The fixed DR fibers are postfixed with 2% osmium tetroxide, dehydrated in graded alcohol series, and embedded in Epon812. Thin sections (1 μm) are cut from each block, stained with alkaline Toluidine blue, and examined by light microscopy. Ultrathin sections (80 nm thick) are cut with an Ultracut S (Leica, Austria), and then stained with uranyl acetate and Lead citrate, respectively. The stained sections are observed under an electron microscope (JEM-1200EX; JEOL, Tokyo, Japan).

### 2) SCI Model

### Anti-LPA Antibody Injection:

The intracerebroventricular (i.c.v. or i.t.) injections of anti-LPA antibody are carried out into the right lateral ventricle of mice. The i.c.v. or i.t. injections are given in a volume of 5 μl (1 μg) 1˜5 times.

### Nociception Test:

The paw pressure test and thermal paw withdrawal test are carried out at 2, 4, 8 and 12 weeks after SCI.

### 3) ICS Model

### Anti-LPA Antibody Injection:

The i.c.v. or i.t. injections of anti-LPA antibody are given in a volume of 5 μl (1 μg) 1˜5 times.

### Nociception Test:

The paw pressure test and thermal paw withdrawal test are carried out at 1, 3, 5, 12, and 19 days after ICS.

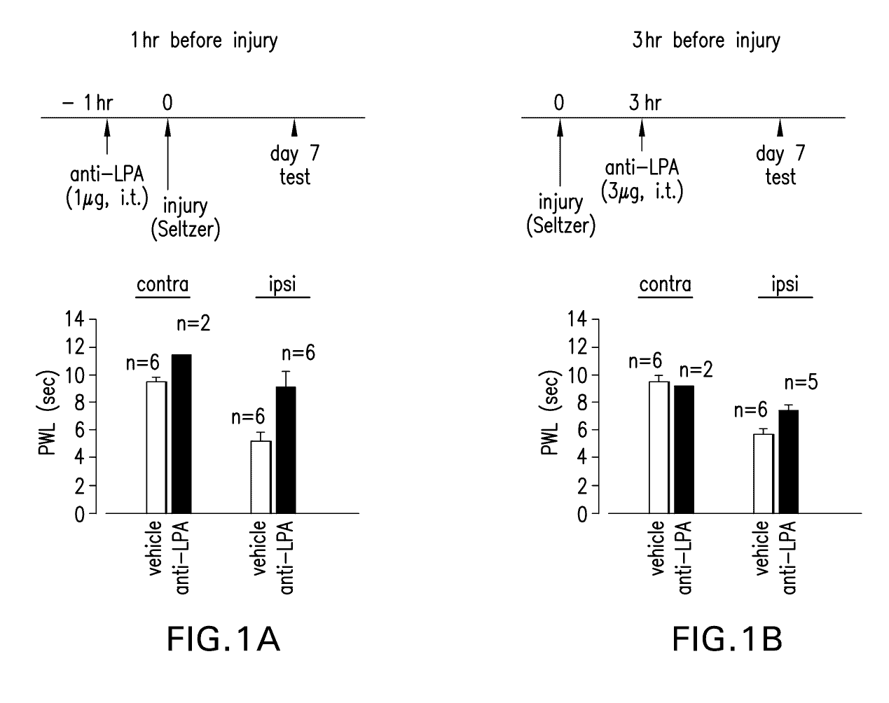

The results of a preliminary PSNI experiment are shown in FIG. 1.

1. Prophylactic experiment (FIG. 1A): Mice were injected with antibody to LPA (B3) (1 ug, intrathecally) one hour before partial sciatic nerve injury, which induces peripheral neuropathic pain [Seltzer, et al. (1990), Pain, 43(2), 205-218]. The injury was performed on only one hindlimb per animal, and pain responses were measured on the injured (ipsilateral) and uninjured (contralateral) sides. The thermal paw withdrawal latency (PWL) test (Hargreaves, et al., 1988) was used to quantitate the pain response. Briefly, a thermal beam was focused on the hind limb foot pads of mice placed on a glass surface and the withdrawal response latency was measured (in seconds). Thus, a higher (longer time) response indicates less pain and a lower (shorter time) response indicates more pain.

FIG. 1A shows that the partial sciatic nerve injury causes a dramatically increased pain response (shortened PWL times) on the injured side (“ipsi”) compared to the uninjured side (“contra”) in the absence of antibody treatment (comparison of the white bars). This effect is prevented by treatment with anti-LPA antibody (B3) (black bars).

2. Interventional experiment (FIG. 1B): Mice were injected with antibody to LPA (B3) (3 ug, intrathecally) three hours after partial sciatic nerve injury (ibid.). FIG. 1B shows that, as above, the partial sciatic nerve injury causes a dramatically increased pain response (shortened PWL times) on the injured side (“ipsi”) compared to the uninjured side (“contra”) in the absence of antibody treatment (comparison of the white bars). This effect is at least partially reversed by treatment post-injury with anti-LPA antibody (B3) (black bars).

### Example 9: Effect of Anti-LPA Antibody in a Rat Model of Adjuvant-Induced Arthritis (AIA)

Inflammation was established in female Lewis rate (150-200 g). 10 animals per dose group were injected subcutaneously in the tail with 3 mg of heat-killed Mycobacterium buytricum suspended in paraffin oil (3 mg/0.150 ml) on day 0. Control animals were injected with paraffin oil only. Rats were assigned numbers and body weights were followed each week. By day 9-10 rats manifested signs of disease and baselines were taken by measuring paw edema (volume) with a plethysmometer. In diseased rats a paw volume of 0.200 ml greater than paw volume in control rats (1.2 ml) was required for inclusion in the study. Animals were randomized based on paw edema and then received vehicle, isotype control, or humanized anti-LPA antibody LT3015. Paw volume was measured seven days and eleven days after antibody dosing. ED₅₀ and ED₈₀ data were calculated based on the data from eleven days following dosing. Vocalization as a measurement of pain was evaluated in the study rats on the final day of the study. Terminal blood collection was performed. Plasma samples were analyzed for antibody concentration. Plasma and paw fluid samples were analyzed for cytokines, eicosanoids, or other inflammatory mediators of interest. Paws were collected at termination for possible histological evaluation.

Dosing: Dosing began once disease manifested, approximately d11-25. Dosing was every 3 days, intraperitoneally, for approximately 5 doses.

Groups: 4 groups=32 rats total, as follows:
1. Negative Control—paraffin oil only—Vehicle
2. Positive Control—Naproxen 10 mg/kg daily, by mouth
3. Isotype Control—40 mg/kg unrelated antibody every 3 days
4. Anti-LPA antibody LT3015—40 mg/kg every 3 days
5. Anti-LPA antibody LT3015—8 mg/kg every 3 days
6. Anti-LPA antibody LT3015—1.6 mg/kg every 3 days.

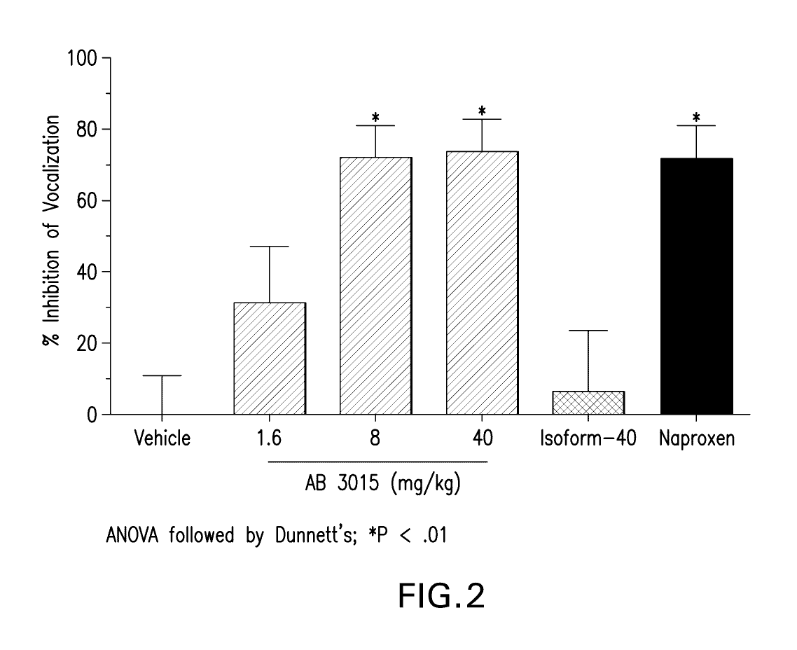

In this study, treatment with humanized anti-LPA antibody (LT3015) was found to reverse the pain vocalization response in the rat AIA model of arthritis. This is shown in FIG. 2. The bars indicate reduction in vocalization. Vehicle alone and isoform control did not decrease vocalization; while the medium (8 mg/kg) and high (40 mg/kg) doses of anti-LPA antibody reduced vocalization by 70-75%, as did the positive control, Naproxen. The low dose (1.6 mg/kg) of LT3015 reduced vocalizations by approximately 30%, showing a dose dependent effect.

### Example 10: Anti-Cancer Activities of Anti-LPA Monoclonal Antibodies

Cancer Cell Proliferation

LPA is a potent growth factor supporting cell survival and proliferation by stimulation of Gᵢ, G_{q} and G_{12/13} via GPCR-receptors and activation of downstream signaling events. Cell lines were tested for their proliferative response to LPA (0.01 mM to 10 mM). Cell proliferation was assayed by using the cell proliferation assay kit from Chemicon (Temecula Calif.) (Panc-1) and the Cell-Blue titer from Pierce (Caki-1). Each data point is the mean of three independent experiments. LPA increased proliferation of 7 human-derived tumor cell lines in a dose dependent manner including SKOV3 and OVCAR3 (ovarian cancer), Panc-1 (pancreatic cancer), Caki-1 (renal carcinoma cell), DU-145 (prostate cancer), A549 (lung carcinoma), and HCT-116 (colorectal adenocarcinoma) cells and one rat-derived tumor cell line, RBL-2H3 (rat leukemia cells). Even though tumor-derived cells normally have high basal levels of proliferation, LPA appears to further augment proliferation in most tumor cell lines. Anti-LPA mAbs (B7 and B58) were assessed for the ability to inhibit LPA-induced proliferation in selected human cancer cell lines. The increase in proliferation induced by LPA was shown to be mitigated by the addition of anti-LPA mAb.

Anti-LPA mAb Sensitizes Tumor Cells to Chemotherapeutic Agents

The ability of LPA to protect ovarian tumor cells against apoptosis when exposed to clinically-relevant levels of the chemotherapeutic agent, paclitaxel (Taxol) was investigated. SKVO3 cells were treated with 1% FBS (S), Taxol (0.5 mM), +/− anti-LPA mAbs for 24 h. LPA protected SKOV3 cells from Taxol-induced apoptosis. Apoptosis was assayed by measurement of the caspase activity as recommended by the manufacturer (Promega). As anticipated, LPA protected most of the cancer cell lines tested from taxol-induced cell death. When the anti-LPA antibody B7 was added to a selection of the LPA responsive cells, it blocked the ability of LPA to protect cells from death induced by the cytotoxic chemotherapeutic agent. Moreover, the anti-LPA antibody was able to remove the protection provided by serum. Serum is estimated to contain about 5-20 uM LPA. Taxol induced caspase-3,7 activation in SKOV3 cells and the addition of serum to cells protected cells from apoptosis. Taxol-induced caspase activation was enhanced by the addition of LT3000 to the culture medium. This suggests that the protective and anti-apoptotic effects of LPA were removed by the selective antibody mediated neutralization of the LPA present in serum.

Anti-LPA mAb Inhibits LPA-Mediated Migration of Tumor Cells

An important characteristic of metastatic cancers is that the tumor cells escape contact inhibition and migrate away from their tissue of origin. LPA has been shown to promote metastatic potential in several cancer cell types. Accordingly, we tested the ability of anti-LPA mAb to block LPA-dependent cell migration in several human cancer cell lines by using the cell monolayer scratch assay. Cells were seeded in 96 well plates and grown to confluence. After 24 h of starvation, the center of the wells was scratched with a pipette tip. In this art-accepted “scratch assay,” the cells respond to the scratch wound in the cell monolayer in a stereotypical fashion by migrating toward the scratch and close the wound. Progression of migration and wound closure are monitored by digital photography at 10× magnification at desired timepoints. Cells were not treated (NT), treated with LPA (2.5 mM) with or w/o mAb B7 (10 μg/ml) or an isotype matching non-specific antibody (NS) (10 μg/ml). In untreated cells, a large gap remains between the monolayer margins following the scratch. LPA-treated cells in contrast, have only a small gap remaining at the same timepoint, and a few cells are making contact across the gap. In cells treated with both LPA and the anti-LPA antibody B7, the gap at this timepoint was several fold larger than the LPA-only treatment although not as large as the untreated control cells. This shows that the anti-LPA antibody had an inhibitory effect on the LPA-stimulated migration of renal cell carcinoma (Caki-1) cells. Similar data were obtained with mAbs B3 and B58. This indicates that the anti-LPA mAb can reduce LPA-mediated migration of cell lines originally derived from metastatic carcinoma.

Anti-LPA mAbs Inhibit Release of Pro-Tumorigenic Cytokines from Tumor Cells

LPA is involved in the establishment and progression of cancer by providing a pro-growth tumor microenvironment and promoting angiogenesis. In particular, increases of the pro-growth factors such as IL-8 and VEGF have been observed in cancer cells. IL-8 is strongly implicated in cancer progression and prognosis. IL-8 may exert its effect in cancer through promoting neovascularization and inducing chemotaxis of neutrophils and endothelial cells. In addition, overexpression of IL-8 has been correlated to the development of a drug resistant phenotype in many human cancer types.

Three anti-LPA mAbs (B3, B7 and B58) were tested for their abilities to reduce in vitro IL-8 production compared to a non-specific antibody (NS). Caki-1 cells were seeded in 96 well plates and grown to confluency. After overnight serum starvation, cells were treated with 18:1 LPA (0.2 mM) with or without anti-LPA mAb B3, B7, B58 or NS (Non-Specific). After 24 h, cultured supernatants of renal cancer cells (Caki-1), treated with or without LPA and in presence of increasing concentrations of the anti-LPA mAbs B3, B7 and B58, were collected and analyzed for IL-8 levels using a commercially available ELISA kit (Human Quantikine Kit, R&D Systems, Minneapolis, Minn.). In cells pre-treated with the anti-LPA mAbs, IL-8 expression was significantly reduced in a dose-dependent manner (from 0.1-30 μg/mL mAb) whereas LPA increased the expression of IL-8 by an average of 100% in non-treated cells. The inhibition of IL-8 release by the anti-LPA mAbs was also observed in other cancerous cell lines such as the pancreatic cell line Panc-1. These data suggest that the blockade of the pro-angiogenic factor release is an additional and potentially important effect of these anti-LPA mAbs.

Anti-LPA mAbs Inhibit Angiogenesis In Vivo

One of the anti-LPA mAbs (B7) was tested for its ability to mitigate angiogenesis in vivo using the Matrigel Plug assay. This assay utilizes Matrigel, a proprietary mixture of tumor remnants including basement membranes derived from murine tumors. When Matrigel, or its derivate growth factor-reduced (GFR) Matrigel, is injected sc into an animal, it solidifies and forms a ‘plug.’ If pro-angiogenic factors are mixed with the matrix prior to placement, the plug will be invaded by vascular endothelial cells which eventually form blood vessels. Matrigel can be prepared either alone or mixed with recombinant growth factors (bFGF, VEGF), or tumor cells and then injected sc in the flanks of 6-week old nude (NCr Nu/Nu) female mice. In this example, Caki-1 (renal carcinoma) cells were introduced inside the Matrigel and are producing sufficient levels of VEGF and/or IL8 and LPA. Matrigel plugs were prepared containing 5×10⁵ Caki-1 cells from mice treated with saline or with 10 mg/kg of anti-LPA mAb-B7, every 3 days starting 1 day prior to Matrigel implantation. Plugs were stained for endothelial CD31, followed by quantitation of the micro-vasculature formed in the plugs. Quantitation data were means+/−SEM of at least 16 fields/section from 3 plugs. The plugs from mice treated with the anti-LPA mAb B7 demonstrated a prominent reduction in blood vessel formation, as assayed by endothelial staining for CD31, compared to the plugs from saline-treated mice. Quantification of stained vessels demonstrates a greater than 50% reduction in angiogenesis in Caki-1-containing plugs from animals treated with mAb B7 compared to saline-treated animals. This was a statistically significant reduction (p<0.05 for mAb B7 vs. Saline as determined by Student's T-test) in tumor cell angiogenesis as a result of anti-LPA mAb treatment.

Anti-LPA mAbs Reduces Tumor Progression in a Murine Model of Metastasis

One important characteristic of tumor progression is the ability of a tumor to metastasize and form secondary tumor nodules at remote sites. In vitro studies described hereinabove have demonstrated the ability of LPA to induce tumor cells to escape contact inhibition and promote migration in a scratch assay for cell motility. In these studies, the anti-LPA mAbs also inhibited LPA's tumor growth promoting effectors. The efficacy of the anti-LPA mAb to inhibit tumor metastasis in vivo was also evaluated. The phenomenon of tumor metastasis has been difficult to mimic in animal models. Many investigators utilize an “experimental” metastasis model in which tumor cells are directly injected into the blood stream.

Blood vessel formation is an integral process of metastasis because an increase in the number of blood vessels means cells have to travel a shorter distance to reach circulation. It is believed that anti-LPA mAb will inhibit in vivo tumor cell metastasis, based on the finding that the anti-LPA mAb can block several integral steps in the metastatic process.

Study: The highly metastatic murine melanoma (B16-F10) was used to examine the therapeutic effect of anti-LPA mAbs on metastasis in vivo. This model has demonstrated to be highly sensitive to cPA inhibitors of autotaxin. 4 week old female (C57BL/6) mice received an injection of B16-F10 murine melanoma tumor cells (100 uL of 5×10⁴ cells/animal) via the tail vein. Mice (10 per group) were administered 25 mg/kg of the anti-LPA mAb (either B3 or B7) or saline every three days by i.p. injection. After 18 days, lungs were harvested and analyzed. The pulmonary organs are the preferred metastatic site of the melanoma cells, and were therefore closely evaluated for metastatic nodules. The lungs were inflated with 10% buffered formalin via the trachea, in order to inflate and fix simultaneously, so that even small foci could be detectable on histological examination. Lungs were separated into five lobes and tumors were categorized by dimension (large >5 mm; medium 1-4 mm; small <1 mm) and counted under a dissecting microscope. Upon examination of the lungs, the number of tumors was clearly reduced in antibody-treated animals. For animals treated with mAb B3, large tumors were reduced by 21%, medium tumors by 17% and small tumors by 22%. Statistical analysis by student's T-test gave a p<0.05 for number of small tumors in animals treated with mAb B3 vs saline.

As shown in the above examples, it has now been shown that the tumorigenic effects of LPA are extended to renal carcinoma (e.g., Caki-1) and pancreatic carcinoma (Panc-1) cell lines. LPA induces tumor cell proliferation, migration and release of pro-angiogenic and/or pro-metastatic agents, such as VEGF and IL-8, in both cell lines. It has now been shown that three high-affinity and specific monoclonal anti-LPA antibodies demonstrate efficacy in a panel of in vitro cell assays and in vivo tumor models of angiogenesis and metastasis.

### Example 11: Lpathomab™ in Cancer and Angiogenesis Models

The pleiotropic effects of LPA suggest that reduced availability (effective concentration) of extracellular LPA will (i) reduce growth, metastasis and angiogenesis of primary tumors and (ii) counter-act LPA's protective anti-apoptotic effect on tumor. Because of Lpathomab™/LT3000's potent and specific binding to LPA, we hypothesized that in vivo treatment of LT3000 in preclinical models of cancer would result in various therapeutic benefits.

Preclinical studies were conducted using a variety of in vitro and in vivo systems, demonstrating that Lpathomab™/LT3000 (administered every 3 days at doses of 10-50 mg/kg) exhibits a profile of activity that is consistent with various mechanisms of action, including:

Inhibition of tumor growth in human tumor xenograft models in vivo;

Reduction in LPA-dependent cell proliferation and invasion of human tumor in vitro;

Reduction in angiogenesis, together with reductions in circulating levels of tumorigenic/angiogenic growth factors including IL6, IL8, GM-CSF, MMP2 in vivo;

Reduced metastatic potential; and

Neutralization of LPA-induced protection against tumor-cell death.

In In Vitro Models:

Reduced proliferation of OVCAR3 ovarian cancer cells;

Neutralization of LPA-induced release of IL-8 from Caki-1, IL-8 and IL-6 from SKOV3 (ovarian) tumor cells in vitro;

Mitigation of LPA's effects in protecting SKOV3 tumor cells from apoptosis (which suggests enhanced efficacy when used in combination with standard chemotherapeutic agents);

Inhibition of LPA-induced tumor cell migration and invasion from chemotherapeutic agents.

In In Vivo Models:

Inhibition of metastasis and progression of orthotopic and subcutaneous human tumors implanted in nude mice;

Reduction of tumor-associated angiogenesis in subcutaneous SKOV3 xenograft models and in prostate DU145 cancer cells;

Neutralization of bFGF- and VEGF-induced angiogenesis in the murine Matrigel plug assay; and

Reduced choroidal neovascularization in a model of laser-induced injury of Bruch's membrane in the eye.

Reduced inflammation and fibrosis with modulation of cytokines and growth factors following bleomycin lung injury;

Further details on efficacy of LT3000 in disease models can be found in, e.g., WO 2008/150841 and corresponding US application US-2009-0136483-A1, both of which are commonly assigned with the instant application and incorporated herein by reference in their entirety.

All of the compositions and methods described and claimed herein can be made and executed without undue experimentation in light of the present disclosure. While the compositions and methods of this invention have been described in terms of preferred embodiments, it will be apparent to those of skill in the art that variations may be applied to the compositions and methods. All such similar substitutes and modifications apparent to those skilled in the art are deemed to be within the spirit and scope of the invention as defined by the appended claims.

All patents, patent applications, and publications mentioned in the specification are indicative of the levels of those of ordinary skill in the art to which the invention pertains. All patents, patent applications, and publications, including those to which priority or another benefit is claimed, are herein incorporated by reference to the same extent as if each individual publication was specifically and individually indicated to be incorporated by reference.

The invention illustratively described herein suitably may be practiced in the absence of any element(s) not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method of treating or preventing a disease or disorder associated with aberrant levels of lysophosphatidic acid (LPA), comprising administering to a subject, optionally a human subject, in need of such treatment an antibody or fragment thereof that binds LPA under physiological conditions in an amount effective to reduce in vivo the effective concentration of LPA, thereby effecting treatment or prevention of the disease or disorder, wherein the antibody or fragment thereof that binds lysophosphatidic acid (LPA) under physiological conditions comprises at least one heavy chain variable domain and at least one light chain variable domain, wherein
(i) each heavy chain variable domain comprises first, second, and third heavy chain complementarity determining regions (CDRs), wherein the first heavy chain CDR comprises an amino acid sequence selected from the group consisting of SEQ ID NO: 1, 7, 8, 17 and 23, the second heavy chain CDR comprises an amino acid sequence selected from the group consisting of SEQ ID NO: 2, 9, 13 and 18, and the third heavy chain CDR comprises an amino acid sequence selected from the group consisting of SEQ ID NO: 3, 10, 14, 16 and 19; and
(ii) each light chain variable domain comprises first, second, and third light chain CDRs, wherein the first light chain CDR comprises an amino acid sequence selected from the group consisting of SEQ ID NO: 4, 11, 15 and 20, the second light chain CDR comprises an amino acid sequence selected from the group consisting of SEQ ID NO: 5, 12 and 21, and the third light chain CDR comprises an amino acid sequence selected from the group consisting of SEQ ID NO: 6 and 22, and
wherein the disease or disorder is selected from the group consisting of a hyperproliferative disease, including cancer; an immune-related disease, including an autoimmune disease, allograft rejection and graft-vs-host disease; obesity; type 2 diabetes; an ocular disease, including macular degeneration; pain; a disease associated with aberrant angiogenesis or neovascularization; apoptosis; fibrogenesis or fibrosis, including scleroderma, pulmonary fibrosis, renal fibrosis, skin fibrosis, cardiac fibrosis, and hepatic fibrosis; wound repair and healing; and spider bite.

2. The method of claim 1 wherein the antibody or fragment thereof is selected from the group consisting of a chimeric antibody, a humanized antibody or a full-length antibody, or an LPA-binding fragment of one of the foregoing.

3. The method of claim 1 wherein the antibody or fragment thereof that binds lysophosphatidic acid (LPA) under physiological conditions comprises two heavy chain variable domains and two light chain variable domains.

4. The method of claim 1 wherein the antibody or fragment thereof is conjugated to a moiety selected from the group consisting of a polymer, a radionuclide, a chemotherapeutic agent, and a detection agent.

5. A method of claim 1 wherein the pain is neuropathic pain.

6. A method of claim 1 wherein the antibody is administered parenterally, intracranially, intrathecally, intra-cerebrospinally, subcutaneously, intra-articularly, intrasynovially, orally, topically, intratracheally or by inhalation.

7. A method of claim 6 wherein parenteral administration is intravenous, intraarterial, subcutaneous, intraperitoneal or intramuscular administration, wherein said administration may be by injection or by infusion.

8. A method of detecting LPA or a metabolite thereof in a sample obtained from a subject, comprising detecting binding of LPA or a metabolite thereof in a sample to an antibody, or antigen-binding fragment thereof, that specifically binds LPA or a metabolite thereof, under conditions that allow the antibody or antigen-binding fragment thereof to bind to the LPA or metabolite thereof if present in the sample, wherein the antibody or antigen-binding fragment thereof comprises at least one immunoglobulin heavy chain variable domain and at least one immunoglobulin light chain variable domain, wherein:
(i) each immunoglobulin heavy chain variable domain comprises first, second, and third heavy chain complementarity determining regions (CDRs), wherein the first heavy chain CDR comprises an amino acid sequence selected from the group consisting of SEQ ID NO: 1, 7, 8, 17 and 23, the second heavy chain CDR comprises an amino acid sequence selected from the group consisting of SEQ ID NO: 2, 9, 13 and 18, and the third heavy chain CDR comprises an amino acid sequence selected from the group consisting of SEQ ID NO: 3, 10, 14, 16 and 19; and
(ii) each immunoglobulin light chain variable domain comprises first, second, and third light chain CDRs, wherein the first light chain CDR comprises an amino acid sequence selected from the group consisting of SEQ ID NO: 4, 11, 15 and 20, the second light chain CDR comprises an amino acid sequence selected from the group consisting of SEQ ID NO; 5, 12 and 21, and the third light chain CDR comprises an amino acid sequence selected from the group consisting of SEQ ID NO: 6 and 22.

9. A method according to claim 8 wherein the sample is an animal-derived sample, and optionally wherein the subject is a mammal, optionally a human.

10. A method according to claim 9 wherein the animal-derived sample is selected from the group consisting of a tissue sample, optionally a tissue biopsy sample, and a bodily fluid sample, optionally wherein the bodily fluid sample is selected from the group consisting of whole blood, plasma, serum, urine, semen, bile, aqueous humor, vitreous humor, mucus, bronchioalveolar lavage fluid, and sputum.

11. A method according to claim 8 wherein the antibody or antigen-binding fragment thereof is a monoclonal antibody or antigen-binding fragment thereof.

12. A method according to claim 8 further comprising measuring an amount of LPA or metabolite thereof in the sample, optionally wherein the method further comprises comparing a level of LPA or metabolite thereof in the sample to a reference level of LPA or metabolite thereof obtained from a normal animal of the same species as the subject, wherein the presence of an increased level of LPA or metabolite thereof relative to the reference level correlates with the presence of disease, optionally wherein the method further comprises comparing a level of LPA or metabolite thereof in the sample to a desired level of LPA or metabolite thereof, and, if necessary, altering a therapeutic dosage of an anti-LPA agent administered to the subject, wherein the anti-LPA agent modulates the effective concentration of LPA, in order to regulate the effective concentration of LPA in the subject.

13. A diagnostic kit for detecting lysophosphatidic acid (LPA) for use in a method according to claim 1, comprising:
(a) a diagnostic reagent comprising a derivatized LPA that comprises a hydrocarbon chain, wherein a carbon atom within the hydrocarbon chain is derivatized with a reactive group; and
(b) an antibody, optionally a monoclonal antibody, or antigen-binding fragment thereof, that specifically binds LPA, wherein the antibody or antigen-binding fragment thereof comprises at least one immunoglobulin heavy chain variable domain and at least one immunoglobulin light chain variable domain, wherein:
(i) each immunoglobulin heavy chain variable domain comprises first, second, and third heavy chain complementarity determining regions (CDRs), wherein the first heavy chain CDR comprises an amino acid sequence selected from the group consisting of SEQ ID NO: 1, 7, 8, 17 and 23, the second heavy chain CDR comprises an amino acid sequence selected from the group consisting of SEQ ID NO: 2, 9, 13 and 18, and the third heavy chain CDR comprises an amino acid sequence selected from the group consisting of SEQ ID NO: 3, 10, 14, 16 and 19; and
(ii) each immunoglobulin light chain variable domain comprises first, second, and third light chain CDRs, wherein the first light chain CDR comprises an amino acid sequence selected from the group consisting of SEQ ID NO: 4, 11, 15 and 20, the second light chain CDR comprises an amino acid sequence selected from the group consisting of SEQ ID NOS, 12 and 21, and the third light chain CDR comprises an amino acid sequence selected from the group consisting of SEQ ID NO: 6 and 22.

14. A diagnostic kit according to claim 13 wherein the reactive group is selected from the group consisting of a sulfhydryl (thiol) group, a carboxylic acid group, a cyano group, an ester, a hydroxy group, an alkene, an alkyne, an acid chloride group, and a halogen atom.

15. A diagnostic kit according to claim 13 wherein the derivatized LPA is associated with a solid support, optionally covalently associated with the solid support.

16. A diagnostic kit according to claim 13 wherein the derivatized LPA is conjugated to a carrier moiety selected from the group consisting of polyethylene glycol, colloidal gold, adjuvant, a silicone bead, and a protein, optionally wherein the protein is selected from the group consisting of keyhole limpet hemocyanin, albumin, bovine thyroglobulin, and soybean trypsin inhibitor, optionally wherein the carrier moiety is associated with a solid support.

17. A diagnostic kit according to claim 13 that is an ELISA kit.
